(19) United States
(12) Patent Application Publication
(10) Pub. No.: US 2023/0190608 A1
(43) Pub. Date: Jun. 22, 2023

(54) COMPOSITIONS AND METHODS FOR IMPROVING THE APPEARANCE OF SKIN

(71) Applicant: L'Oreal, Paris (FR)

(72) Inventors: I-Chien LIAO, Princeton, NJ (US); Charbel BOUEZ, Hoboken, NJ (US); Ying CHEN, Montville, NJ (US); Qian ZHENG, Bridgewater, NJ (US); Julien LABOUREAU, Chevilly-Larue (FR); Ronni Lynn WEINKAUF, Oradell, NJ (US)

(21) Appl. No.: 18/087,192

(22) Filed: Dec. 22, 2022

Related U.S. Application Data
(60) Provisional application No. 63/293,012, filed on Dec. 22, 2021.

(30) Foreign Application Priority Data
Apr. 1, 2022 (FR) 2202983

(51) Int. Cl. A61K 8/44; A61K 8/67; A61K 8/365; A61Q 19/08

(52) U.S. Cl. CPC A61K 8/44; A61K 8/671; A61K 8/365; A61Q 19/08

(57) ABSTRACT

The disclosure relates to compositions, methods, and kits for improving the appearance of the skin by applying acetyl trifluoromethylphenyl valylglycine, at least one retinoid, and optionally at least one additional skin peeling agent to the skin.

### TECHNICAL FIELD

The disclosure relates to compositions for treating skin, methods for treating skin with skin treatment compositions, and kits containing skin treatment compositions.

### BACKGROUND

Aging can result in the appearance of wrinkles and other changes to the skin. The aging appearance of skin is caused, in part, by the disorganization of elastin and collagen fibers, which leads to a loss of elasticity, flexibility, and firmness.

Retinol is used to treat aging skin. Once oxidized in the skin into retinoic acid, the biologically active molecule can stimulate new collagen production in the dermis, while reducing matrix metalloproteinase (MMP) activity, inducing epidermal hyperplasia, enhancing exfoliation, and decreasing melanin synthesis. However, retinol use can also result in skin irritation, such as redness, dryness, and inflammation. In addition, the stability of retinol decreases over time.

Desquamation is a natural phenomenon that slows or reduces the effects of aging on the skin. It is related to the fact that the epidermis, which constitutes the upper layer of the skin, is in constant regeneration. The epidermis is made up of several cell layers, of which the deepest is the basal layer made up of undifferentiated cells. Over time, these cells will differentiate and migrate to the surface of the epidermis constituting the different layers of the epidermis, until the corneocytes (dead cells) form an outermost layer, the corneal layer (also called the stratum corneum), or the surface of the epidermis, which are dead cells that are removed by desquamation. This loss in surface is compensated by the migration of cells from the basal layer towards the surface of the epidermis resulting in perpetual skin renewal.

Forced removal of the corneal layer can accelerate desquamation, accelerate the renewal of the skin's surface, slow the effects of aging of skin, and/or improve the appearance of skin. It is known that skin peeling agents can remove the corneal layer. The term “peeling” conventionally denotes a controlled action of irritation of the surface of the skin with a chemical substance or via a physical process. A surface peeling treatment targets the epidermis and is based on a surface attack of the skin. It affects the epidermis either partially or entirely. A peeling treatment may consist, for example, in applying to the skin a chemical substance (a “chemical peeling agent”) for the purpose of bringing about limited and controlled destruction of the epidermis (and in some embodiments of the upper layers of the dermis). The treatment may require multiple applications, and the whole procedure may take anywhere from one or a few days to several weeks or months.

Consumers seeking antiaging treatments desire easy-use and efficacious skincare routines that yield rapid visible results with good tolerance and minimal downtime. Thus, there is an ongoing need for improved skin treatments that can increase skin renewal, while reducing the side effects of retinoids and peeling agents, particularly retinoids. It has now been surprisingly and unexpectedly discovered that acetyl trifluoromethylphenyl valylglycine works synergistically with retinol and optionally peeling agents to improve skin renewal, thereby improving the appearance of the skin, while reducing skin irritation associated with, e.g., retinoid use.

### SUMMARY

The instant disclosure relates to compositions and methods for treating skin, for example by stimulating, increasing, and/or improving skin renewal and/or anti-aging benefits. For example, the disclosed treatments may work synergistically to encourage or improve skin turnover, all while reducing irritation from retinoids, and imparting broader and/or stronger anti-aging and skin quality benefits to skin and/or achieving these benefits with faster kinetics as compared retinol alone or peptide and chemical peel use alone. According to certain aspects of the disclosure, methods and skin treatment compositions may advantageously enhance skin elasticity and/or reduce the appearance of discoloration/hyperpigmentation and/or wrinkles, such as under eye wrinkles, fine lines, and/or nasolabial folds.

In various embodiments, the disclosure relates to a two-phase method for treating skin, the method comprising a phase (1) comprising applying to the skin (a) acetyl trifluoromethylphenyl valylglycine at least one time per day; (b) at least one retinoid or derivative thereof at least one time every two to three days; and (c) optionally at least one additional skin peeling agent at least one time every two to three days, wherein the (b) at least one retinoid or derivative thereof and the (c) optional at least one additional skin peeling agent are separately applied to the skin at least about 6 hours, preferably at least about 12 hours, more preferably at least about 18 hours, most preferably at least about 24 hours apart; and wherein phase (1) lasts for a period of time of at least about 10 days, preferably at least about 14 days, more preferably at least about 21 days, most preferably at least about 28 days; and a phase (2) comprising applying to the skin (a) acetyl trifluoromethylphenyl valylglycine at least one time per day; and (b) at least one retinoid or derivative thereof at least one time every two to three days, wherein phase (2) lasts for a period of time of at least about 5 days, preferably at least about 7 days, more preferably at least about 10 days, most preferably at least about 14 days.

In further embodiments, the disclosure relates to a two-phase method for treating skin, the method comprising a phase (1) comprising separately applying to the skin (a) a composition comprising acetyl trifluoromethylphenyl valylglycine, at least one time per day; (b) a composition comprising at least one retinoid or derivative thereof, at least one time every two to three days; and (c) optionally a composition comprising at least one additional skin peeling agent, at least one time every two to three days, wherein the (b) composition comprising at least one retinoid or derivative thereof and the (c) optional composition comprising at least one additional skin peeling agent are applied to the skin at least about 6 hours, preferably at least about 12 hours, more preferably at least about 18 hours, most preferably at least about 24 hours apart; and wherein phase (1) lasts for a period of time of at least about 10 days, preferably at least about 14 days, more preferably at least about 21 days, most preferably at least about 28 days; and a phase (2) comprising separately applying to the skin (a) a composition comprising acetyl trifluoromethylphenyl valylglycine, at least one time per day; and (b) a composition comprising at least one retinoid or derivative thereof, at least one time every two to three days, wherein phase (2) lasts for a period of time of at least about 5 days, preferably at least about 7 days, more preferably at least about 10 days, most preferably at least about 14 days.

In yet further embodiments, the disclosure relates to a two-phase method for treating skin, the method comprising a phase (1) comprising separately applying to the skin (a) a composition comprising acetyl trifluoromethylphenyl valylglycine, at least one time per day; (b) a composition comprising at least one retinoid or derivative thereof and acetyl trifluoromethylphenyl valylglycine, at least one time every two to three days; and (c) optionally a composition comprising at least one additional skin peeling agent, at least one time every two to three days, wherein the (b) composition comprising at least one retinoid or derivative thereof and acetyl trifluoromethylphenyl valylglycine and the (c) optional composition comprising at least one additional skin peeling agent are applied to the skin at least about 6 hours, preferably at least about 12 hours, more preferably at least about 18 hours, most preferably at least about 24 hours apart; and wherein phase (1) lasts for a period of time of at least about 10 days, preferably at least about 14 days, more preferably at least about 21 days, most preferably at least about 28 days; and a phase (2) comprising separately applying to the skin (a) a composition comprising acetyl trifluoromethylphenyl valylglycine, at least one time per day; and (b) a composition comprising at least one retinoid or derivative thereof and acetyl trifluoromethylphenyl valylglycine, at least one time every two to three days, wherein phase (2) lasts for a period of time of at least about 5 days, preferably at least about 7 days, more preferably at least about 10 days, most preferably at least about 14 days.

In still further embodiments, the disclosure relates to a two-phase method for treating skin, the method comprising a phase (1) comprising applying to the skin (a) from about 0.1 to about 10 wt % of acetyl trifluoromethylphenyl valylglycine, at least one time, preferably at least two times, per day; (b) from about 0.001 to about 10 wt % of at least one retinoid or derivative thereof, at least one time every two to three days; and (c) optionally from about 0.1 to about 25 wt % of at least one additional skin peeling agent chosen from α-hydroxy acids, β-hydroxy acids, or mixtures thereof, at least one time every two to three days, wherein the (b) at least one retinoid or derivative thereof and the (c) optional at least one additional skin peeling agent are separately applied to the skin at least about 6 hours, preferably at least about 12 hours, more preferably at least about 18 hours, most preferably at least about 24 hours apart; and wherein phase (1) lasts for a period of time of at least about 10, preferably at least about 14 days, more preferably at least about 21 days, most preferably at least about 28 days; and a phase (2) comprising applying to the skin (a) from about 0.1 to about 10 wt % of acetyl trifluoromethylphenyl valylglycine, at least one time, preferably at least two times, per day; and (b) from about 0.001 to about 10 wt % of at least one retinoid or derivative thereof, at least one time every two to three days, wherein phase (2) lasts for a period of time of at least about 5, preferably at least about 7, more preferably at least about 10, most preferably at least about 14 days, and wherein amounts of all components are relative to the total weight of the composition in which it is present.

The methods may, for example, be methods for reducing the appearance of nasolabial folds, methods for reducing the appearance of wrinkles in skin, methods for improving the appearance of skin, and/or methods for reducing the appearance of discoloration of skin. In further embodiments, the disclosure relates to improving tolerance of the skin to retinoids and/or retinoid derivatives, comprising applying to the skin acetyl trifluoromethylphenyl valylglycine at least one retinoid or derivative thereof.

The disclosure also relates to compositions comprising acetyl trifluoromethylphenyl valylglycine and at least one retinoid or derivative thereof or at least one skin peeling agent other than retinoids or derivatives thereof.

The disclosure also relates to kits comprising acetyl trifluoromethylphenyl valylglycine, at least one retinoid or derivative thereof, and optionally at least one skin peeling agent and/or implementation for exfoliation of skin.

### BRIEF DESCRIPTION OF THE FIGURES

The accompanying drawings, which are incorporated herein and constitute part of this specification, illustrate exemplary embodiments of the disclosure, and, together with the general description given above and the description provided herein, serve to explain features of the disclosure.

FIG. 1 shows two-dimensional ex-vivo histological cross-sections of (1) a skin sample treated with vehicle control (Control—2 wt % ethanol), (2) a skin sample treated with 0.1 wt % acetyl trifluoromethylphenyl valylglycine (ATV), (3) a skin sample treated with 0.003% retinol, and (4) a skin sample treated with 0.003% retinol and 0.1 wt % acetyl trifluoromethylphenyl valylglycine. The cross-sections are stained for (A) hematoxylin and eosin (H&E) expression, (B) Filaggrin (FLG) expression, and (C) Desmocollin-1 (DSC) expression. Black scale bars=100 μm.

FIG. 2 shows the amount of a 2% solution of acetyl trifluoromethylphenyl valylglycine in the stratum corneum (SC), the epidermis (Ep), the dermis (D), the receptor fluid (RF), and the amount that passes through the stratum corneum into the skin (Ep+D+RF), after application to skin samples using a Franz cell horizontal cell clamp system.

FIG. 3 shows (A) a home-care product regimen for 3 study groups (Groups 1-3), and (B) treatment phases for Groups 2 and 3 of Example 3.

FIG. 4 shows a chart comparing the clinical outcomes of a 2 week, 6 week, and 12 week regimen described in Example 3, with Group 1 undergoing treatment with 0.3% retinol and Group 3 undergoing treatment with 2% solution of acetyl trifluoromethylphenyl valylglycine, 0.3% retinol, and a 0.45% salicylic acid/11.11% lactic acid chemical peel. Checkmarks (“√”) denote a statistically significant improvement in outcomes for Group 3 over Group 1. Equals signs (“=”) denote no statistical difference in outcomes between Group 1 and Group 3.

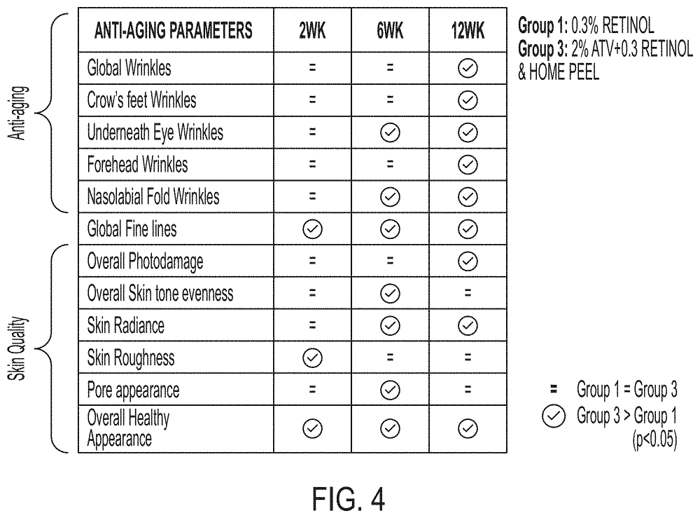

FIG. 5 shows a chart comparing the clinical outcomes of a 2 week, 6 week, and 12 week regimen described in Example 3, with Group 1 undergoing treatment with 0.3% retinol and Group 3 undergoing treatment with 2% solution of acetyl trifluoromethylphenyl valylglycine, 0.3% retinol, and a 0.45% salicylic acid/11.11% lactic acid chemical peel as compared to baseline. Blank boxes denote no statistical difference in clinical outcomes for Group 1 or Group 3 over baseline. Boxes with solid diagonal lines denote statistically significant (p<0.05) improvements in clinical outcomes for Group 1 or Group 3 over baseline. Boxes with dashed diagonal lines denote statistically significant and clinically significant (p<0.05) improvements in outcomes for Group 1 or Group 3 over baseline.

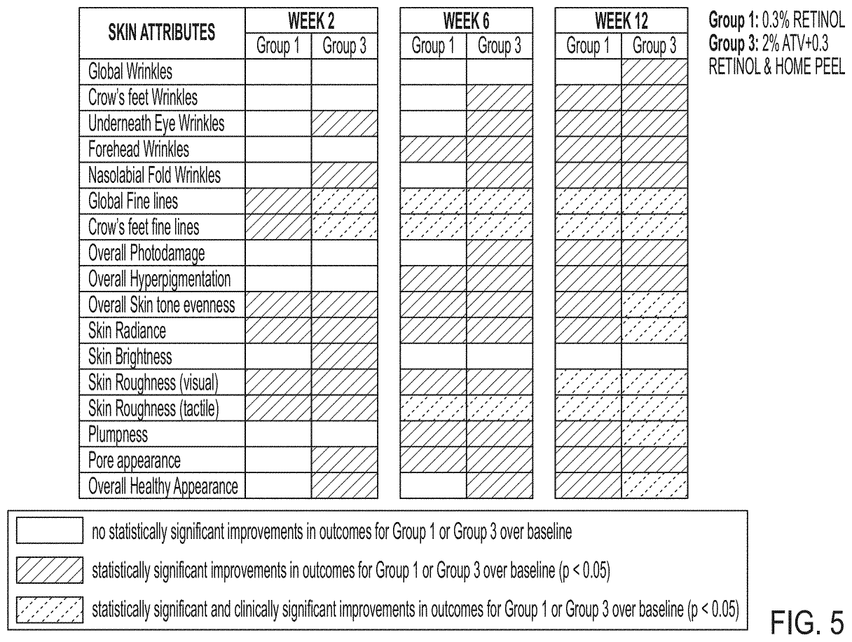

FIG. 6 shows a graph of clinical grading for global fine lines between all treatment groups (Groups 1-3) over the 12-week regimen of Example 3. An asterisk (“*”) denotes a statistically significant difference (p<0.05) in global fine lines compared to Group 1 at the respective timepoint. A hash (“#”) denotes a statistically significant difference (p<0.05) in global fine lines compared to Group 2 at the respective timepoint.

FIG. 7 shows a graph of clinical grading for nasolabial folds between all treatment groups (Groups 1-3) over the 12-week regimen of Example 3. An asterisk (“*”) denotes a statistically significant difference (p<0.05) in nasolabial folds compared to Group 1 at the respective timepoint.

FIG. 8 shows a graph of clinical grading for overall photodamage between all treatment groups (Groups 1-3) over the 12-week regimen of Example 3. An asterisk (“*”) denotes a statistically significant difference (p<0.05) in photodamage compared to Group 1 at the respective timepoint. A hash (“#”) denotes a statistically significant difference (p<0.05) in photodamage compared to Group 2 at the respective timepoint.

FIG. 9 shows a graph of clinical grading for under eye wrinkles between all treatment groups (Groups 1-3) over the 12-week regimen of Example 3. An asterisk (“*”) denotes a statistically significant difference (p<0.05) in under eye wrinkles compared to Group 1 at the respective timepoint. A hash (“#”) denotes a statistically significant difference (p<0.05) in under eye wrinkles compared to Group 2 at the respective timepoint.

FIG. 10 shows a graph of clinical grading for overall healthy appearance between all treatment groups (Groups 1-3) over the 12-week regimen of Example 3. An asterisk (“*”) denotes a statistically significant difference (p<0.05) in overall healthy appearance compared to Group 1 at the respective timepoint. A hash (“#”) denotes a statistically significant difference (p<0.05) in overall healthy appearance compared to Group 2 at the respective timepoint.

FIG. 11 shows a graph of clinical grading for global fine lines between treatment Group 1 (Retinol) and Group 3 (ATV+Peel+Retinol) over the 12-week regimen of Example 3. An asterisk (“*”) denotes a statistically significant difference (p<0.05) in global fine lines compared to Group 1 at the respective timepoint.

FIG. 12 shows a graph of clinical grading for nasolabial folds between treatment Group 1 (Retinol) and Group 3 (ATV+Peel+Retinol) over the 12-week regimen of Example 3. An asterisk (“*”) denotes a statistically significant difference (p<0.05) in nasolabial folds compared to Group 1 at the respective timepoint.

FIG. 13 shows a graph of clinical grading for photodamage between treatment Group 1 (Retinol) and Group 3 (ATV+Peel+Retinol) over the 12-week regimen of Example 3. An asterisk (“*”) denotes a statistically significant difference (p<0.05) in photodamage compared to Group 1 at the respective timepoint.

FIG. 14 shows a graph of clinical grading for undereye wrinkles between treatment Group 1 (Retinol) and Group 3 (ATV+Peel+Retinol) over the 12-week regimen of Example 3. An asterisk (“*”) denotes a statistically significant difference (p<0.05) in under eye wrinkles compared to Group 1 at the respective timepoint.

FIG. 15 shows a graph of clinical grading for overall healthy appearance between treatment Group 1 (Retinol) and Group 3 (ATV+Peel+Retinol) over the 12-week regimen of Example 3. An asterisk (“*”) denotes a statistically significant difference (p<0.05) in overall healthy appearance compared to Group 1 at the respective timepoint.

### DETAILED DESCRIPTION

The disclosure relates to compositions, methods, and kits for treating skin, such as by increasing, accelerating, and/or improving skin renewal, and/or improving the appearance of skin. The compositions according to various aspects of the disclosure comprise acetyl trifluoromethylphenyl valylglycine and/or at least one retinoid or derivative thereof, and optionally at least one additional skin peeling agent. The methods according to various aspects of the disclosure comprise applying to skin, simultaneously or sequentially, acetyl trifluoromethylphenyl valylglycine, at least one retinoid or derivative thereof, and optionally at least one additional skin peeling agent. Kits according to various aspects of the disclosure comprise acetyl trifluoromethylphenyl valylglycine, at least one retinoid or derivative thereof, and optionally at least one additional skin peeling agent.

The compositions and methods according to various aspects of the disclosure may, in various embodiments, protect against and/or slow degradation of skin elastase, improve skin elasticity, decrease sagging, improve or accelerate skin turnover/desquamation of the skin, stimulate or improve epidermal renewal, increase exfoliation, reduce or slow the appearance of skin aging (such as wrinkles), improve skin discoloration and/or pigmentation, increase skin keratinization or upper terminal differentiation, and/or may smooth skin. For example, the skin treatment methods generally include applying acetyl trifluoromethylphenyl valylglycine, at least one retinoid or derivative thereof, and optionally at least one additional skin peeling agent to skin to accelerate skin renewal/turnover and/or anti-aging benefits, while reducing irritation from retinoids. Surprisingly, it has been found that in some embodiments, the skin treatment methods and compositions according to the disclosure impart broader and/or stronger anti-aging and skin quality benefits to skin, and/or achieve these benefits with faster kinetics as compared to peptide, retinol, and/or chemical peel use alone.

In various embodiments, acetyl trifluoromethylphenyl valylglycine, the at least one retinoid or derivative thereof, and the at least one additional skin peeling agent work synergistically to improve the appearance of the skin. For example, in various embodiments the skin treatment methods and compositions disclosed herein may be effective in reducing the appearance of wrinkles by the acceleration of cell turnover, for example, by keratinization and upper terminal differentiation. In further embodiments, the skin treatment methods and compositions disclosed herein reduce the appearance of wrinkles by the inhibition of elastase, thereby allowing for the growth of elastin fibers as opposed to their breakdown by these enzymes, improving skin elasticity. In yet further embodiments, the skin treatment methods and compositions disclosed herein reduce skin irritation from retinoids.

A more detailed description of compositions, methods, and kits for treating skin is provided below.

### I. Compositions

Compositions according to various embodiments of the disclosure comprise acetyl trifluoromethylphenyl valylglycine, at least one retinoid or derivative thereof, and/or at least one additional skin peeling agent. In certain embodiments, compositions according to the disclosure comprise both acetyl trifluoromethylphenyl valylglycine and at least one retinoid or derivative thereof. In other embodiments, compositions according to the disclosure comprise acetyl trifluoromethylphenyl valylglycine, at least one retinoid or derivative thereof, and at least one additional skin peeling agent.

### Acetyl Trifluoromethylphenyl Valylglycine

According to various embodiments, compositions according to the disclosure comprise at least about 0.01 wt % of acetyl trifluoromethylphenyl valylglycine, such as from about 0.01 to about 25 wt %, relative to the total weight of the composition in which it is present. For example, the amount of acetyl trifluoromethylphenyl valylglycine may range from about 0.1 to about 25 wt %, about 0.1 to about 22 wt %, about 0.1 to about 19 wt %, about 0.1 to about 16 wt %, about 0.1 to about 14 wt %, about 0.1 to about 12 wt %, about 0.1 to about 10 wt %, about 0.1 to about 8 wt %, about 0.1 to about 6 wt %, about 0.1 to about 5 wt %, about 0.1 to about 4 wt %, about 0.1 to about 3 wt %, about 0.1 to about 2 wt %, about 0.1 to about 1 wt %; about 0.25 to about 25 wt %, about 0.25 to about 22 wt %, about 0.25 to about 19 wt %, about 0.25 to about 16 wt %, about 0.25 to about 14 wt %, about 0.25 to about 12 wt %, about 0.25 to about 10 wt %, about 0.25 to about 8 wt %, about 0.25 to about 6 wt %, about 0.25 to about 5 wt %, about 0.25 to about 4 wt %, about 0.25 to about 3 wt %, about 0.25 to about 2 wt %, about 0.25 to about 1 wt %; about 0.5 to about 25 wt %, about 0.5 to about 22 wt %, about 0.5 to about 19 wt %, about 0.5 to about 16 wt %, about 0.5 to about 14 wt %, about 0.5 to about 12 wt %, about 0.5 to about 10 wt %, about 0.5 to about 8 wt %, about 0.5 to about 6 wt %, about 0.5 to about 5 wt %, about 0.5 to about 4 wt %, about 0.5 to about 3 wt %, about 0.5 to about 2 wt %, about 0.5 to about 1 wt %; about 0.75 to about 25 wt %, about 0.75 to about 22 wt %, about 0.75 to about 19 wt %, about 0.75 to about 16 wt %, about 0.75 to about 14 wt %, about 0.75 to about 12 wt %, about 0.75 to about 10 wt %, about 0.75 to about 8 wt %, about 0.75 to about 6 wt %, about 0.75 to about 5 wt %, about 0.75 to about 4 wt %, about 0.75 to about 3 wt %, about 0.75 to about 2 wt %; about 1 to about 25 wt %, about 1 to about 22 wt %, about 1 to about 19 wt %, about 1 to about 16 wt %, about 1 to about 14 wt %, about 1 to about 12 wt %, about 1 to about 10 wt %, about 1 to about 8 wt %, about 1 to about 6 wt %, about 1 to about 5 wt %, about 1 to about 4 wt %, about 1 to about 3 wt %, about 1 to about 2 wt %; about 2 to about 25 wt %, about 2 to about 22 wt %, about 2 to about 19 wt %, about 2 to about 16 wt %, about 2 to about 14 wt %, about 2 to about 12 wt %, about 2 to about 10 wt %, about 2 to about 8 wt %, about 2 to about 6 wt %, about 2 to about 5 wt %, about 2 to about 4 wt %, about 2 to about 3 wt %; about 5 to about 25 wt %, about 5 to about 22 wt %, about 5 to about 19 wt %, about 5 to about 16 wt %, about 5 to about 14 wt %, about 5 to about 12 wt %, about 5 to about 10 wt %, about 5 to about 8 wt %; about 7.5 to about 27.5 wt %, about 7.5 to about 22 wt %, about 7.5 to about 19 wt %, about 7.5 to about 16 wt %, about 7.5 to about 14 wt %, about 7.5 to about 12 wt %, about 7.5 to about 10 wt %; about 10 to about 27.5 wt %, about 10 to about 22 wt %, about 10 to about 19 wt %, about 10 to about 16 wt %, about 10 to about 14 wt %, about 10 to about 12 wt %; about 15 to about 27.5 wt %, about 15 to about 22 wt %, about 15 to about 19 wt %; about 20 to about 27.5 wt %, or about 20 to about 22 wt %, including ranges and subranges therebetween, relative to the total weight of the composition in which it is present.

In some embodiments, the composition comprises an amount of acetyl trifluoromethylphenyl valylglycine such that about 0.001 to about 2.5 wt % of acetyl trifluoromethylphenyl valylglycine penetrates through the epidermis and reaches the dermal layer of the skin. For example, the compositions may comprise an amount of acetyl trifluoromethylphenyl valylglycine such that about 0.002 to about 2 wt %, about 0.002 to about 1.9 wt %, about 0.002 to about 1.8 wt %, about 0.002 to about 1.7 wt %, about 0.002 to about 1.6 wt %, about 0.002 to about 1.5 wt %, about 0.002 to about 1.4 wt %, about 0.002 to about 1.3 wt %, about 0.002 to about 1.2 wt %, about 0.002 to about 1.1 wt %, about 0.002 to about 1 wt %, about 0.002 to about 0.9 wt %, about 0.002 to about 0.8 wt %, about 0.002 to about 0.7 wt %, about 0.002 to about 0.6 wt %, about 0.002 to about 0.5 wt %, about 0.002 to about 0.4 wt %, about 0.002 to about 0.3 wt %, about 0.002 to about 0.2 wt %, about 0.002 to about 0.1 wt %, about 0.002 to about 0.09 wt %, about 0.002 to about 0.08 wt %, about 0.002 to about 0.07 wt %, about 0.002 to about 0.06 wt %, about 0.002 to about 0.05 wt %, about 0.0042 to about 0.04 wt %, about 0.002 to about 0.03 wt %, about 0.002 to about 0.02 wt %, about 0.002 to about 0.01 wt %, about 0.006 to about 2 wt %, about 0.006 to about 1.9 wt %, about 0.006 to about 1.8 wt %, about 0.006 to about 1.7 wt %, about 0.006 to about 1.6 wt %, about 0.006 to about 1.5 wt %, about 0.006 to about 1.4 wt %, about 0.006 to about 1.3 wt %, about 0.006 to about 1.2 wt %, about 0.006 to about 1.1 wt %, about 0.006 to about 1 wt %, about 0.006 to about 0.9 wt %, about 0.006 to about 0.8 wt %, about 0.006 to about 0.7 wt %, about 0.006 to about 0.6 wt %, about 0.006 to about 0.5 wt %, about 0.006 to about 0.4 wt %, about 0.006 to about 0.3 wt %, about 0.006 to about 0.2 wt %, about 0.006 to about 0.1 wt %, about 0.006 to about 0.09 wt %, about 0.006 to about 0.08 wt %, about 0.006 to about 0.07 wt %, about 0.006 to about 0.06 wt %, about 0.006 to about 0.05 wt %, about 0.006 to about 0.04 wt %, about 0.006 to about 0.03 wt %, about 0.006 to about 0.02 wt %, about 0.006 to about 0.01 wt %, about 0.008 to about 2 wt %, about 0.008 to about 1.9 wt %, about 0.008 to about 1.8 wt %, about 0.008 to about 1.7 wt %, about 0.008 to about 1.6 wt %, about 0.008 to about 1.5 wt %, about 0.008 to about 1.4 wt %, about 0.008 to about 1.3 wt %, about 0.008 to about 1.2 wt %, about 0.008 to about 1.1 wt %, about 0.008 to about 1 wt %, about 0.008 to about 0.9 wt %, about 0.008 to about 0.8 wt %, about 0.008 to about 0.7 wt %, about 0.008 to about 0.6 wt %, about 0.008 to about 0.5 wt %, about 0.008 to about 0.4 wt %, about 0.008 to about 0.3 wt %, about 0.008 to about 0.2 wt %, about 0.008 to about 0.1 wt %, about 0.008 to about 0.09 wt %, about 0.008 to about 0.08 wt %, about 0.008 to about 0.07 wt %, about 0.008 to about 0.06 wt %, about 0.008 to about 0.05 wt %, about 0.008 to about 0.04 wt %, about 0.008 to about 0.03 wt %, about 0.008 to about 0.02 wt %, about 0.008 to about 0.01 wt %, about 0.01 to about 2 wt %, about 0.01 to about 1.9 wt %, about 0.01 to about 1.8 wt %, about 0.01 to about 1.7 wt %, about 0.01 to about 1.6 wt %, about 0.01 to about 1.5 wt %, about 0.01 to about 1.4 wt %, about 0.01 to about 1.3 wt %, about 0.01 to about 1.2 wt %, about 0.01 to about 1.1 wt %, about 0.01 to about 1 wt %, about 0.01 to about 0.9 wt %, about 0.01 to about 0.8 wt %, about 0.01 to about 0.7 wt %, about 0.01 to about 0.6 wt %, about 0.01 to about 0.5 wt %, about 0.01 to about 0.4 wt %, about 0.01 to about 0.3 wt %, about 0.01 to about 0.2 wt %, about 0.01 to about 0.1 wt %, about 0.01 to about 0.09 wt %, about 0.01 to about 0.08 wt %, about 0.01 to about 0.07 wt %, about 0.01 to about 0.06 wt %, about 0.01 to about 0.05 wt %, about 0.01 to about 0.04 wt %, about 0.01 to about 0.03 wt %, about 0.01 to about 0.02 wt %, about 0.025 to about 2 wt %, about 0.025 to about 1.9 wt %, about 0.025 to about 1.8 wt %, about 0.025 to about 1.7 wt %, about 0.025 to about 1.6 wt %, about 0.025 to about 1.5 wt %, about 0.025 to about 1.4 wt %, about 0.025 to about 1.3 wt %, about 0.025 to about 1.2 wt %, about 0.025 to about 1.1 wt %, about 0.025 to about 1 wt %, about 0.025 to about 0.9 wt %, about 0.025 to about 0.8 wt %, about 0.025 to about 0.7 wt %, about 0.025 to about 0.6 wt %, about 0.025 to about 0.5 wt %, about 0.025 to about 0.4 wt %, about 0.025 to about 0.3 wt %, about 0.025 to about 0.2 wt %, about 0.025 to about 0.1 wt %, about 0.025 to about 0.09 wt %, about 0.025 to about 0.08 wt %, about 0.025 to about 0.07 wt %, about 0.025 to about 0.06 wt %, about 0.025 to about 0.05 wt %, about 0.025 to about 0.04 wt %, about 0.05 to about 2 wt %, about 0.05 to about 1.9 wt %, about 0.05 to about 1.8 wt %, about 0.05 to about 1.7 wt %, about 0.05 to about 1.6 wt %, about 0.05 to about 1.5 wt %, about 0.05 to about 1.4 wt %, about 0.05 to about 1.3 wt %, about 0.05 to about 1.2 wt %, about 0.05 to about 1.1 wt %, about 0.05 to about 1 wt %, about 0.05 to about 0.9 wt %, about 0.05 to about 0.8 wt %, about 0.05 to about 0.7 wt %, about 0.05 to about 0.6 wt %, about 0.05 to about 0.5 wt %, about 0.05 to about 0.4 wt %, about 0.05 to about 0.3 wt %, about 0.05 to about 0.2 wt %, about 0.05 to about 0.1 wt %, about 0.05 to about 0.09 wt %, about 0.05 to about 0.08 wt %, about 0.05 to about 0.07 wt %, about 0.06 to about 2 wt %, about 0.06 to about 1.9 wt %, about 0.06 to about 1.8 wt %, about 0.06 to about 1.7 wt %, about 0.06 to about 1.6 wt %, about 0.06 to about 1.5 wt %, about 0.06 to about 1.4 wt %, about 0.06 to about 1.3 wt %, about 0.06 to about 1.2 wt %, about 0.06 to about 1.1 wt %, about 0.06 to about 1 wt %, about 0.06 to about 0.9 wt %, about 0.06 to about 0.8 wt %, about 0.06 to about 0.7 wt %, about 0.06 to about 0.6 wt %, about 0.06 to about 0.5 wt %, about 0.06 to about 0.4 wt %, about 0.06 to about 0.3 wt %, about 0.06 to about 0.2 wt %, about 0.06 to about 0.1 wt %, about 0.06 to about 0.09 wt %, about 0.06 to about 0.08 wt %, about 0.06 to about 0.07 wt %, about 0.07 to about 2 wt %, about 0.07 to about 1.9 wt %, about 0.07 to about 1.8 wt %, about 0.07 to about 1.7 wt %, about 0.07 to about 1.6 wt %, about 0.07 to about 1.5 wt %, about 0.07 to about 1.4 wt %, about 0.07 to about 1.3 wt %, about 0.07 to about 1.2 wt %, about 0.07 to about 1.1 wt %, about 0.07 to about 1 wt %, about 0.07 to about 0.9 wt %, about 0.07 to about 0.8 wt %, about 0.07 to about 0.7 wt %, about 0.07 to about 0.6 wt %, about 0.07 to about 0.5 wt %, about 0.07 to about 0.4 wt %, about 0.07 to about 0.3 wt %, about 0.07 to about 0.2 wt %, about 0.07 to about 0.1 wt %, about 0.07 to about 0.09 wt %, about 0.07 to about 0.08 wt %, about 0.08 to about 2 wt %, about 0.08 to about 1.9 wt %, about 0.08 to about 1.8 wt %, about 0.08 to about 1.7 wt %, about 0.08 to about 1.6 wt %, about 0.08 to about 1.5 wt %, about 0.08 to about 1.4 wt %, about 0.08 to about 1.3 wt %, about 0.08 to about 1.2 wt %, about 0.08 to about 1.1 wt %, about 0.08 to about 1 wt %, about 0.08 to about 0.9 wt %, about 0.08 to about 0.8 wt %, about 0.08 to about 0.7 wt %, about 0.08 to about 0.6 wt %, about 0.08 to about 0.5 wt %, about 0.08 to about 0.4 wt %, about 0.08 to about 0.3 wt %, about 0.08 to about 0.2 wt %, about 0.08 to about 0.1 wt %, about 0.08 to about 0.09 wt %, about 0.09 to about 2 wt %, about 0.09 to about 1.9 wt %, about 0.09 to about 1.8 wt %, about 0.09 to about 1.7 wt %, about 0.09 to about 1.6 wt %, about 0.09 to about 1.5 wt %, about 0.09 to about 1.4 wt %, about 0.09 to about 1.3 wt %, about 0.09 to about 1.2 wt %, about 0.09 to about 1.1 wt %, about 0.09 to about 1 wt %, about 0.09 to about 0.9 wt %, about 0.09 to about 0.8 wt %, about 0.09 to about 0.7 wt %, about 0.09 to about 0.6 wt %, about 0.09 to about 0.5 wt %, about 0.09 to about 0.4 wt %, about 0.09 to about 0.3 wt %, about 0.09 to about 0.2 wt %, about 0.09 to about 0.1 wt %, about 0.1 to about 2 wt %, about 0.1 to about 1.9 wt %, about 0.1 to about 1.8 wt %, about 0.1 to about 1.7 wt %, about 0.1 to about 1.6 wt %, about 0.1 to about 1.5 wt %, about 0.1 to about 1.4 wt %, about 0.1 to about 1.3 wt %, about 0.1 to about 1.2 wt %, about 0.1 to about 1.1 wt %, about 0.1 to about 1 wt %, about 0.1 to about 0.9 wt %, about 0.1 to about 0.8 wt %, about 0.1 to about 0.7 wt %, about 0.1 to about 0.6 wt %, about 0.1 to about 0.5 wt %, about 0.1 to about 0.4 wt %, about 0.1 to about 0.3 wt %, or about 0.1 to about 0.2 wt % of acetyl trifluoromethylphenyl valylglycine penetrates the epidermis and reaches the dermal layer of the skin.

In some embodiments, the compositions comprise an amount of acetyl trifluoromethylphenyl valylglycine such that about 0.01 to about 1.5 wt %, preferably about 0.025 to about 1.3 wt %, more preferably about 0.5 to about 1.1 wt %, most preferably about 0.6 to about 1 wt % of acetyl trifluoromethylphenyl valylglycine penetrates the epidermis and reaches the dermal layer of the skin.

### Retinoids

According to various embodiments, compositions according to the disclosure comprise at least one retinoid or derivative thereof. As used herein, a “retinoid” refers to a compound that comprises a four-isoprenoid unit. Exemplary and non-limiting retinoids or derivatives thereof that may be used include retinol (vitamin A), retinal, retiferol, tretinoin (all-trans-retinoic acid, e.g. retinoic acid, Retin-A), isotretinoin, alitretinoin (9-cis-retinoic acid), etretinate, acitretin, tazarotene, bexarotene, Adapalene, N,N-diethylaminoethyl 13-cis-retinoate, 13-cis-retinoic acid, N,N-diethylaminoethyl all-trans-retinoate, retinyl N,N-dimethyl-2-aminoacetate, retinyl palmitate, retinoid metabolites, or agents that can be metabolized into a retinoid or a retinoid metabolite.

According to various embodiments, compositions according to the disclosure comprise at least about 0.00001 wt % of the at least one retinoid or derivative thereof, such as from about 0.0001 to about 10 wt %, about 0.0002 to about 9.7 wt %, about 0.0003 to about 9.4 wt %, about 0.0004 to about 9 wt %, about 0.0005 to about 8.7 wt %, about 0.0006 to about 8.4 wt %, about 0.0007 to about 8 wt %, about 0.0008 to about 7.7 wt %, about 0.0009 to about 7.4 wt %, about 0.001 to about 7 wt %, about 0.002 to about 6.7 wt %, about 0.003 to about 6.4 wt %, about 0.004 to about 6 wt %, about 0.005 to about 5.7 wt %, about 0.006 to about 5.4 wt %, about 0.007 to about 5 wt %, about 0.008 to about 4.7 wt %, about 0.009 to about 4.4 wt %, about 0.01 to about 4 wt %, about 0.02 to about 3.7 wt %, about 0.02 to about 3.4 wt %, about 0.03 to about 3 wt %, about 0.04 to about 2.7 wt %, about 0.05 to about 2.4 wt %, about 0.06 to about 2 wt %, about 0.07 to about 1.7 wt %, about 0.08 to about 1.4 wt %, about 0.09 to about 1 wt %, about 0.1 to about 1 wt %, about 0.2 to about 1 wt %, about 0.3 to about 1 wt %, about 0.4 to about 1 wt %, about 0.5 to about 1 wt %, about 0.6 to about 1 wt %, about 0.7 to about 1 wt %, about 0.8 to about 1 wt %, about 0.9 to about 1 wt % relative to the total weight of the composition in which it is present including all ranges and subranges therebetween, relative to the total weight of the composition in which it is present. For example, the amount of the at least one retinoid or derivative thereof may range from about 0.0003 to about 9.5 wt %, about 0.0005 to about 9 wt %, about 0.0007 to about 8.5 wt %, about 0.0009 to about 8 wt %, about 0.001 to about 7 wt %, including all ranges and subranges therebetween, relative to the total weight of the composition in which it is present.

According to various embodiments, the acetyl trifluoromethylphenyl valylglycine and at least one retinoid or derivative may be present in the same composition, or may be present in separate compositions. The compositions comprising acetyl trifluoromethylphenyl valylglycine and/or at least one retinoid or derivative can be, for example, topical skin treatment composition(s). The topical skin treatment composition(s) can be formulated as a cream, a lotion, a serum, a gel, or an ampoule, or any other topically suitable form. For example, the skin treatment compositions may be in the form of water-in-oil emulsions, oil-in-water emulsions, silicone-in-water emulsions, water-in-silicone emulsions, or combinations thereof. In an embodiment, the acetyl trifluoromethylphenyl valylglycine and optionally the at least one retinoid or derivative thereof is present in an emulsion, such as an oil-in-water emulsion. For example, the emulsion can be an oil/silicone in water emulsion. In certain embodiments, the emulsion comprises a silicone oil such as dimethicone. The emulsion may also optionally comprise emulsifiers, such as octyldodecyl xyloside and/or polysorbate.

### Additional Skin Peeling Agents

According to various embodiments, compositions according to the disclosure optionally comprise at least one additional physical and/or chemical skin peeling agent. In various embodiments, compositions may comprise one or more physical peeling agents, e.g. microbeads or other small particles. In further embodiments, compositions may comprise one or more chemical peeling agents.

Chemical skin peeling agents that can be used include but are not limited to β-hydroxyacids (BHAs), for example salicylic acid and derivatives thereof (including n-octanoyl 5-salicylic acid, otherwise known as capryloyl salicylic acid (INCI name)); α-hydroxyacids (AHAs), such as glycolic, citric, lactic, tartric, malic, or mandelic acids; 8-hexadecene-1,16-dicarboxylic acid, or 9-octadecene dioic acid; urea and derivatives thereof; trichloroacetic acid; gentisic acid and derivatives thereof; oligofuctoses; cinnamic acid; extract of Saphora japonica; resveratrol; certain derivatives of jasmonic acid; resorcinol; phenol; or mixtures thereof.

Further exemplary and non-limiting chemical skin peeling agents that may be used include compounds involved in peeling or degrading corneodesmosomes, such as aminosulfonic compounds, e.g. 4-(2-hydroxyethyl)piperazine-1-propanesulfonic acid (HEPES); 2-oxothiazolidine-4-carboxylic acid (procysteine) and derivatives thereof; the derivatives of α-amino acids of the glycine type (as described in EP-0 852 949) and also sodium methyl glycine diacetate sold by BASF under the trade name Trilon M; honey; derivatives of sugar such as O-octanoyl-6-D-maltose and N-acetyl glucosamine; or mixtures thereof.

As still further exemplary and non-limiting chemical skin peeling agents suitable for use in compositions according to the disclosure, mention be made of EDTA and derivatives thereof, laminaria extracts, O-linoleyl-6-D-gluocose; (3-hydroxy pentylcyclopentyl) acetic acid, glycerol trilactate, S carboxymethyl cysteine, silica-containing derivatives of salicylate such as those described in patent EP 0 796 861, oligofuctases such as those described in patent EP 0 218 200, agents having effects on transglutaminase as in patent EP 0 899 330; extract of the flower ficus opuntia indica such as Exfolactive® from Silab; 8-hexadecene 1,16-dicarboxylic acid; esters of glucose and of vitamin F; or mixtures thereof.

The chemical skin peeling agent(s) may, in certain embodiments, be chosen from α-hydroxy acids such as citric, lactic, glycolic, malic, tartric, or mandelic acids; β-hydroxy acids such as salicylic acid or derivatives thereof, e.g. n-octanoyl-5-salicylic acid; tricholoacetic acid; or mixtures thereof. In at least some embodiments, the compositions comprise lactic acid, salicylic acid, or mixtures thereof, and in one preferred embodiment the compositions comprise lactic acid and optionally salicylic acid as additional skin peeling agents.

The total amount of additional chemical skin peeling agent(s) present in the composition may range from about 0.01 to about 25 wt %, such as from about 0.1 to about 20 wt %, relative to the total weight of the composition in which it is present. For example, the total amount of chemical peeling agent present in the composition may range from about 0.5 to about 25 wt %, about 0.5 to about 22 wt %, about 0.5 to about 19 wt %, about 0.5 to about 16 wt %, about 0.5 to about 14 wt %, about 0.5 to about 12 wt %, about 0.5 to about 10 wt %, about 0.5 to about 8 wt %, about 0.5 to about 6 wt %, about 0.5 to about 5 wt %, about 0.5 to about 4 wt %, about 0.5 to about 3 wt %, about 0.5 to about 2 wt %, about 0.5 to about 1 wt %; about 1 to about 25 wt %, about 1 to about 22 wt %, about 1 to about 19 wt %, about 1 to about 16 wt %, about 1 to about 14 wt %, about 1 to about 12 wt %, about 1 to about 10 wt %, about 1 to about 8 wt %, about 1 to about 6 wt %, about 1 to about 5 wt %, about 1 to about 4 wt %, about 1 to about 3 wt %, about 1 to about 2 wt %; about 2 to about 25 wt %, about 2 to about 22 wt %, about 2 to about 19 wt %, about 2 to about 16 wt %, about 2 to about 14 wt %, about 2 to about 12 wt %, about 2 to about 10 wt %, about 2 to about 8 wt %, about 2 to about 6 wt %, about 2 to about 5 wt %, about 2 to about 4 wt %, about 2 to about 3 wt %; about 3 to about 25 wt %, about 3 to about 22 wt %, about 3 to about 19 wt %, about 3 to about 16 wt %, about 3 to about 14 wt %, about 3 to about 12 wt %, about 3 to about 10 wt %, about 3 to about 8 wt %, about 3 to about 6 wt %, about 3 to about 5 wt %, about 3 to about 4 wt %, about 4 to about 25 wt %, about 4 to about 22 wt %, about 4 to about 19 wt %, about 4 to about 16 wt %, about 4 to about 14 wt %, about 4 to about 12 wt %, about 4 to about 10 wt %, about 4 to about 8 wt %, about 4 to about 6 wt %, about 4 to about 5 wt %, about 5 to about 25 wt %, about 5 to about 22 wt %, about 5 to about 19 wt %, about 5 to about 16 wt %, about 5 to about 14 wt %, about 5 to about 12 wt %, about 5 to about 10 wt %, about 5 to about 8 wt %; about 7.5 to about 25 wt %, about 7.5 to about 22 wt %, about 7.5 to about 19 wt %, about 7.5 to about 16 wt %, about 7.5 to about 14 wt %, about 7.5 to about 12 wt %, about 7.5 to about 10 wt %; about 10 to about 25 wt %, about 10 to about 22 wt %, about 10 to about 19 wt %, about 10 to about 16 wt %, about 10 to about 14 wt %, about 10 to about 12 wt %; about 12 to about 25 wt %, about 12 to about 22 wt %, about 12 to about 19 wt %, about 12 to about 16 wt %, about 12 to about 14 wt %, about 15 to about 25 wt %, about 15 to about 22 wt %, about 15 to about 19 wt %, about 17 to about 25 wt %, about 17 to about 22 wt %, or about 17 to about 19 wt %, including ranges and subranges therebetween, relative to the total weight of the composition in which it is present.

For example, in various embodiments, compositions according to the disclosure comprise at least one AHA as a chemical peeling agent. In certain embodiments, the compositions may comprise a total amount of AHAs ranging from about 1 to about 20 wt %, about 1 to about 19 wt %, about 1 to about 18 wt %, about 1 to about 17 wt %, about 1 to about 16 wt %, about 1 to about 15 wt %, about 1 to about 14 wt %, about 1 to about 13 wt %, about 1 to about 12 wt %, about 1 to about 11 wt %, about 1 to about 10 wt %, about 1 to about 9 wt %, about 1 to about 8 wt %, about 1 to about 7 wt %, about 1 to about 6 wt %, about 1 to about 5 wt %, about 1 to about 4 wt %, about 1 to about 3 wt %, about 1 to about 2 wt %, about 2 to about 20 wt %, about 2 to about 19 wt %, about 2 to about 18 wt %, about 2 to about 17 wt %, about 2 to about 16 wt %, about 2 to about 15 wt %, about 2 to about 14 wt %, about 2 to about 13 wt %, about 2 to about 12 wt %, about 2 to about 11 wt %, about 2 to about 10 wt %, about 2 to about 9 wt %, about 2 to about 8 wt %, about 2 to about 7 wt %, about 2 to about 6 wt %, about 2 to about 5 wt %, about 2 to about 4 wt %, about 2 to about 3 wt %, about 3 to about 20 wt %, about 3 to about 19 wt %, about 3 to about 18 wt %, about 3 to about 17 wt %, about 3 to about 16 wt %, about 3 to about 15 wt %, about 3 to about 14 wt %, about 3 to about 13 wt %, about 3 to about 12 wt %, about 3 to about 11 wt %, about 3 to about 10 wt %, about 3 to about 9 wt %, about 3 to about 8 wt %, about 3 to about 7 wt %, about 3 to about 6 wt %, about 3 to about 5 wt %, about 3 to about 4 wt %, about 10 to about 20 wt %, about 10 to about 19 wt %, about 10 to about 18 wt %, about 10 to about 17 wt %, about 10 to about 16 wt %, about 10 to about 15 wt %, about 10 to about 14 wt %, about 10 to about 13 wt %, about 10 to about 12 wt %, about 11 to about 20 wt %, about 11 to about 19 wt %, about 11 to about 18 wt %, about 11 to about 17 wt %, about 11 to about 16 wt %, about 11 to about 15 wt %, about 11 to about 14 wt %, about 11 to about 13 wt %, about 12 to about 20 wt %, about 12 to about 19 wt %, about 12 to about 18 wt %, about 12 to about 17 wt %, about 12 to about 16 wt %, about 12 to about 15 wt %, about 12 to about 14 wt %, about 13 to about 20 wt %, about 13 to about 19 wt %, about 13 to about 18 wt %, about 13 to about 17 wt %, about 13 to about 16 wt %, or about 13 to about 15 wt %, including ranges and subranges therebetween, relative to the total weight of the composition in which it is present. In certain embodiments, the total amount of AHAs ranges from about 2 to about 4 wt %, about 4 to about 7 wt %, or about 13 to about 16 wt %, relative to the total weight of the composition in which it is present. In other embodiments, the total amount of AHAs is about 3 wt %, is between 5 and 6 wt %, or is between 14 and 15 wt %, relative to the total weight of the composition in which it is present.

In certain embodiments, compositions according to the disclosure comprise at least one BHA as a chemical peeling agent. For example, the compositions may comprise a total amount of BHAs ranging from about 0.01 to about 2 wt %, about 0.01 to about 1.9 wt %, about 0.01 to about 1.8 wt %, about 0.01 to about 1.7 wt %, about 0.01 to about 1.6 wt %, about 0.01 to about 1.5 wt %, about 0.01 to about 1.4 wt %, about 0.01 to about 1.3 wt %, about 0.01 to about 1.2 wt %, about 0.01 to about 1.1 wt %, about 0.01 to about 1.0 wt %, about 0.01 to about 0.9 wt %, about 0.01 to about 0.8 wt %, about 0.01 to about 0.7 wt %, about 0.01 to about 0.6 wt %, about 0.01 to about 0.5 wt %, about 0.01 to about 0.4 wt %, about 0.01 to about 0.3 wt %, about 0.01 to about 0.2 wt %, about 0.01 to about 0.1 wt %, about 0.05 to about 2 wt %, about 0.05 to about 1.9 wt %, about 0.05 to about 1.8 wt %, about 0.05 to about 1.7 wt %, about 0.05 to about 1.6 wt %, about 0.05 to about 1.5 wt %, about 0.05 to about 1.4 wt %, about 0.05 to about 1.3 wt %, about 0.05 to about 1.2 wt %, about 0.05 to about 1.1 wt %, about 0.05 to about 1.0 wt %, about 0.05 to about 0.9 wt %, about 0.05 to about 0.8 wt %, about 0.05 to about 0.7 wt %, about 0.05 to about 0.6 wt %, about 0.05 to about 0.5 wt %, about 0.05 to about 0.4 wt %, about 0.05 to about 0.3 wt %, about 0.05 to about 0.2 wt %, about 0.05 to about 0.1 wt %, about 0.1 to about 2 wt %, about 0.1 to about 1.9 wt %, about 0.1 to about 1.8 wt %, about 0.1 to about 1.7 wt %, about 0.1 to about 1.6 wt %, about 0.1 to about 1.5 wt %, about 0.1 to about 1.4 wt %, about 0.1 to about 1.3 wt %, about 0.1 to about 1.2 wt %, about 0.1 to about 1.1 wt %, about 0.1 to about 1.0 wt %, about 0.1 to about 0.9 wt %, about 0.1 to about 0.8 wt %, about 0.1 to about 0.7 wt %, about 0.1 to about 0.6 wt %, about 0.1 to about 0.5 wt %, about 0.1 to about 0.4 wt %, about 0.1 to about 0.3 wt %, or about 0.1 to about 0.2 wt %, including ranges and subranges therebetween, relative to the total weight of the composition in which it is present. In certain embodiments, the total amount of BHAs is about 0.05 wt %, about 0.06 wt %, about 0.07 wt %, about 0.08 wt %, about 0.09 wt %, about 0.1 wt %, about 0.11 wt %, about 0.12 wt %, about 0.13 wt %, about 0.14 wt %, about 0.15 wt %, about 0.16 wt %, about 0.17 wt %, about 0.18 wt %, about 0.19 wt %, about 0.2 wt %, about 0.21 wt %, about 0.22 wt %, about 0.23 wt %, about 0.24 wt %, or about 0.25 wt %, relative to the total weight of the composition in which it is present.

In certain embodiments, compositions according to the disclosure comprise trichloroacetic acid as a chemical peeling agent. For example, the compositions may comprise a total amount of trichloroacetic acid ranging from about 1 to about 25 wt %, about 1 to about 24 wt %, about 1 to about 23 wt %, about 1 to about 22 wt %, about 1 to about 21 wt %, about 1 to about 20 wt %, about 1 to about 19 wt %, about 1 to about 18 wt %, about 1 to about 17 wt %, about 1 to about 16 wt %, about 1 to about 15 wt %, about 1 to about 14 wt %, about 1 to about 13 wt %, about 1 to about 12 wt %, about 1 to about 11 wt %, about 1 to about 10 wt %, about 1 to about 9 wt %, about 1 to about 8 wt %, about 1 to about 7 wt %, about 1 to about 6 wt %, about 1 to about 5 wt %, about 1 to about 4 wt %, about 1 to about 3 wt %, about 1 to about 2 wt %, about 3 to about 25 wt %, about 3 to about 24 wt %, about 3 to about 23 wt %, about 3 to about 22 wt %, about 3 to about 21 wt %, about 3 to about 20 wt %, about 3 to about 19 wt %, about 3 to about 18 wt %, about 3 to about 17 wt %, about 3 to about 16 wt %, about 3 to about 15 wt %, about 3 to about 14 wt %, about 3 to about 13 wt %, about 3 to about 12 wt %, about 3 to about 11 wt %, about 3 to about 10 wt %, about 3 to about 9 wt %, about 3 to about 8 wt %, about 3 to about 7 wt %, about 3 to about 6 wt %, about 3 to about 5 wt %, 5 to about 25 wt %, about 5 to about 24 wt %, about 5 to about 23 wt %, about 5 to about 22 wt %, about 5 to about 21 wt %, about 5 to about 20 wt %, about 5 to about 19 wt %, about 5 to about 18 wt %, about 5 to about 17 wt %, about 5 to about 16 wt %, about 5 to about 15 wt %, about 5 to about 14 wt %, about 5 to about 13 wt %, about 5 to about 12 wt %, about 5 to about 11 wt %, about 5 to about 10 wt %, about 5 to about 9 wt %, about 5 to about 8 wt %, about 5 to about 7 wt %, 8 to about 25 wt %, about 8 to about 24 wt %, about 8 to about 23 wt %, about 8 to about 22 wt %, about 8 to about 21 wt %, about 8 to about 20 wt %, about 8 to about 19 wt %, about 8 to about 18 wt %, about 8 to about 17 wt %, about 8 to about 16 wt %, about 8 to about 15 wt %, about 8 to about 14 wt %, about 8 to about 13 wt %, about 8 to about 12 wt %, about 8 to about 11 wt %, about 8 to about 10 wt %, about 10 to about 25 wt %, about 10 to about 24 wt %, about 10 to about 23 wt %, about 10 to about 22 wt %, about 10 to about 21 wt %, about 10 to about 20 wt %, about 10 to about 19 wt %, about 10 to about 18 wt %, about 10 to about 17 wt %, about 10 to about 16 wt %, about 10 to about 15 wt %, about 10 to about 14 wt %, about 10 to about 13 wt %, about 10 to about 12 wt %, 12 to about 25 wt %, about 12 to about 24 wt %, about 12 to about 23 wt %, about 12 to about 22 wt %, about 12 to about 21 wt %, about 12 to about 20 wt %, about 12 to about 19 wt %, about 12 to about 18 wt %, about 12 to about 17 wt %, about 12 to about 16 wt %, about 12 to about 15 wt %, or about 12 to about 14 wt %, relative to the total weight of the composition in which it is present.

Optionally, the compositions may comprise more than one skin peeling agent, such as, for example, at least two skin peeling agents, at least three skin peeling agents, at least four skin peeling agents, and so on. For example, the compositions may comprise two or more AHAs, may comprise two or more BHAs, may comprise a combination of one or more AHAs and one or more BHAs, and so on. By way of non-limiting example only, the compositions may comprise glycolic acid and lactic acid, may comprise glycolic acid and salicylic acid, may comprise lactic acid and salicylic acid, may comprise glycolic acid and trichloroacetic acid, may comprise lactic acid and trichloroacetic acid, may comprise salicylic acid and trichloroacetic acid, may comprise glycolic acid, lactic acid, and trichloroacetic acid, may comprise glycolic acid, salicylic acid, and trichloroacetic acid, and so on.

In various embodiments, compositions useful in methods and/or kits according to the disclosure comprise at least one skin peeling agent but are free or substantially free of acetyl trifluoromethylphenyl valylglycine and/or retinoids or derivative thereof.

By way of non-limiting example only, compositions comprising at least one skin peeling agent may comprise additional components and amounts thereof typically used in chemical peeling compositions, such as, for example, at least one solvent, at least one oil, and additional optional components. Such compositions may, in certain embodiments, have a pH not less than about 3.0, such as, for example, not less than about 3.5. The pH can be adjusted to the desired value by addition of a base (organic or inorganic) to the composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid such as a carboxylic acid, for example citric acid.

For example, the compositions comprising at least one skin peeling agent that are free or substantially free of acetyl trifluoromethylphenyl valylglycine may comprise at least one solvent chosen from water and organic solvents. By way of example, the compositions may comprise water in an amount of at least about 15 wt %, such as, for example, from about 20 wt % to about 70 wt %, from about 25 wt % to about 60 wt %, from about 30 wt % to about 50 wt %, or from about 35 wt % to about 40 wt %, including all ranges and subranges thereof.

In further embodiments, the compositions comprising at least one skin peeling agent that are free or substantially free of acetyl trifluoromethylphenyl valylglycine and/or retinoids or derivative thereof include one or more organic solvents such as monoalcohols and polyols, for example ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. In some embodiments, the alcohol solvent is selected from ethanol, isopropyl alcohol, ethylene glycol, propylene glycol, butylene glycol, hexylene glycol, propanediol, glycerin, or mixtures thereof. Optionally, in some embodiments the organic solvent(s) may be present in amounts up to about 20 wt %, such as from about 0.1 to about 20 wt %, from about 1 to about 20 wt %, from about 5 to about 20 wt %, or from about 10 to about 15 wt %, based on the weight of the composition.

In various embodiments, the compositions comprising at least one skin peeling agent that are free or substantially free of acetyl trifluoromethylphenyl valylglycine and/or retinoids or derivative thereof include one or more oils. Optionally, the at least one oil may be immiscible in water. For example, the at least one oil may be selected from hydrocarbons, silicones, fatty alcohols, glycols, and vegetable oils. The at least one oil may include one or a combination of polar and non-polar oil. In some embodiments, the oil may be chosen from hydrocarbon-based oils from plants or of plant origin, mineral oil, ester oils, fatty alcohols containing from 12 to 26 carbon atoms, fatty acids containing from 12 to 26 carbon atoms and vinylpyrrolidone copolymers, and mixtures thereof. In some embodiments, an oil may be chosen from purcellin oil (cetostearyl octanoate), squalane, hemisqualane, isononyl isononanoate, C12 to C15 alkyl benzoate, 2-ethylhexyl palmitate, isodecyl neopentanoate, tridecyl neopentanoate, octyldodecyl neopentanoate, 2-octyldodecyl stearate, 2-octyldodecyl erucate, oleyl erucate, isostearyl isostearate, 2-octyldodecyl benzoate, alcohol or polyalcohol octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate, 2-octyldodecyl myristate and 2-diethylhexyl succinate, cocoglyceride, cyclomethicone, dimethicone, dicaprylyl carbonate, dicapryl maleate, caprylic/capric triglyceride, isopropyl myristate, octyl stearate, isostearyl linoleate, lanolin oil, coconut oil, cocoa butter, olive oil, avocado oil, aloe extracts, jojoba oil, castor oil, fatty acid, oleic acid, stearic acid, fatty alcohol, cetyl alcohol, hexadecyl alcohol, diisopropyl adipate, hydroxybenzoate esters, benzoic acid esters of C9-C15 alcohols, alkanes, mineral oil, silicone, dimethyl polysiloxane, ether, polyoxypropylene butyl ether, polyoxypropylene cetyl ether, C12-C15 alkyl benzoate, aryl alkyl benzoate, Isopropyl Lauroyl sarcosinate, and any combinations thereof.

In some embodiments, the oil is chosen from C13-15 alkane (hemisqualane), dicaprylyl carbonate, isononyl isononanoate, isopropyl myristate, dimethicone, squalane, mineral oil, or mixtures thereof. In one exemplary and non-limiting embodiment, the oil comprises one or a combination of C13-15 alkane (hemisqualane), dicaprylyl carbonate, isononyl isononanoate, isopropyl myristate, dimethicone, squalane, and mineral oil. In some embodiments, the oil includes isopropyl myristate.

In various embodiments, the total amount of one oil present in the compositions comprising at least one skin peeling agent that are free or substantially free of acetyl trifluoromethylphenyl valylglycine and/or retinoids or derivative thereof is at least about 5 wt %, at least about 10 wt %, at least about 15 wt %, or at least about 20 wt %, for example can range from about 5 wt % to about 70 wt %, from about 10 wt % to about 65 wt %, from about 15 wt % to about 60 wt %, or from about 20 wt % to about 55 wt %, including all ranges and subranges thereof, based on the weight of the composition. In one embodiment, the total amount of oil ranges from about 20 wt % to about 60 wt % of the composition.

The compositions comprising at least one skin peeling agent that are free or substantially free of acetyl trifluoromethylphenyl valylglycine and/or retinoids or derivative thereof can also comprise one or more additional optional components typically used in chemical peel compositions, for example one or more additives chosen from citric acid, sodium chloride, chelating agents, antimicrobial agents, neutralizing agents, vitamins (e.g. tocopherol), fragrances, pearlescent agents, odor absorbers, coloring materials, essential oils, fruit extracts, or combinations thereof.

In various embodiments, additional optional components may include one or a combination of antimicrobial agents and their salts, selected from, for example, chlorphenesin, caprylyl glycol, phenoxyethanol, caprylhydroxamic acid, benzoic acid, salicylic acid, benzyl alcohol, phenethyl alcohol, benzalkonium chloride, 4-hydroxyacetophenone, piroctone olamine, hexyl glycerin, ethylhexylglycerin, octylglycerin, benzylglycerin, 3-heptoyl-2,2-propandiol, 1,2-hexandiol, pentylene glycol, or mixtures thereof.

In various embodiments, the amount of one or more additional optional components, alone or in combination, may be present in the compositions comprising at least one skin peeling agent that are free or substantially free of acetyl trifluoromethylphenyl valylglycine and/or retinoids or derivative thereof in an amount ranging from about 0.001 wt % to about 20 wt %, such as from about 0.005 wt % to about 0.01 wt %, from about 0.01 wt % to about 0.1 wt %, from about 0.15 wt % to about 5 wt %, from about 0.40 wt % to about 4 wt %, from about 0.5 wt % to about 2.5 wt % by weight, or from about 1 wt % to about 2 wt %, including all ranges and subranges thereof, based on the weight of the composition.

### II. Methods

Methods according to the disclosure comprise applying (a) acetyl trifluoromethylphenyl valylglycine and (b) at least one retinoid or derivative thereof, and optionally (c) at least one additional skin peeling agent, to the skin. The at least one additional skin peeling agent is not limited, and can be chosen from, for example, dermabrasion, microdermabrasion, physical exfoliators, and/or chemical agents. The methods may be, for example, methods for reducing the appearance of nasolabial folds, methods for reducing the appearance of wrinkles in skin, methods for improving the appearance of skin, and/or methods for reducing the appearance of discoloration of skin.

In various embodiments, the acetyl trifluoromethylphenyl valylglycine and at least one retinoid or derivative thereof can be applied to the skin simultaneously (e.g. when both are present in one composition or when each is present in separate compositions that are mixed at or near the time of use) or consecutively (e.g. when each is present in separate compositions that are applied sequentially to the skin). The skin may be, for example, skin on the face, neck, chest, or hands.

In certain embodiments, the methods comprise applying an amount of a composition comprising acetyl trifluoromethylphenyl valylglycine to the skin, such that about 0.1 gram or more of acetyl trifluoromethylphenyl valylglycine is applied to the skin. For example, the composition may be applied such that about 0.05 gram per decimeter (g/dm²) or more, such as about 0.06 g/dm² or more, such as about 0.07 g/dm² or more, such as about 0.08 g/dm² or more, such as about 0.09 g/dm² or more, such as about 0.1 g/dm² or more, such as about 0.15 g/dm² or more, about 0.2 g/dm² or more, about 0.3 g/dm² or more, about 0.4 g/dm² or more, about 0.5 g/dm² or more, about 0.75 g/dm² or more, about 1 g/dm² or more, about 1.5 g/dm² or more, about 2 g/dm² or more, about 2.5 g/dm² or more, or about 3 g/dm² or more of acetyl trifluoromethylphenyl valylglycine is applied to the skin. In some embodiments, the composition is applied such that about 0.05 to about 10 g/dm², about 0.06 to about 9 g/dm², about 0.07 to about 8 g/dm², about 0.08 to about 7 g/dm², about 0.09 to about 6 g/dm², about 0.1 to about 5 g/dm², about 0.15 to about 4 g/dm², about 0.2 to about 3 g/dm², about 0.3 to about 2 g/dm², about 0.4 to about 1 g/dm²; about 0.5 to about 10 g/dm², about 0.5 to about 9 g/dm², about 0.5 to about 8 g/dm², about 0.5 to about 7 g/dm², about 0.5 to about 6 g/dm², about 0.5 to about 5 g/dm², about 0.5 to about 4 g/dm², about 0.5 to about 3 g/dm², about 0.5 to about 2 g/dm², about 0.5 to about 1 g/dm²; or about 1 to about 10 g/dm², about 1 to about 9 g/dm², about 1 to about 8 g/dm², about 1 to about 7 g/dm², about 1 to about 6 g/dm², about 1 to about 5 g/dm², about 1 to about 4 g/dm², about 1 to about 3 g/dm², or about 1 to about 2 g/dm² of acetyl trifluoromethylphenyl valylglycine is applied to the skin.

In some embodiments, the acetyl trifluoromethylphenyl valylglycine can be left on the skin for at least 10 seconds, at least 20 seconds, at least 30 seconds, at least 40 seconds, at least 50 seconds, at least 1 minute, at least 2 minutes, at least 3 minutes, at least 5 minutes, at least 10 minutes, at least 15 minutes, at least 20 minutes, at least 25 minutes, at least 30 minutes, at least 35 minutes, at least 40 minutes, at least 45 minutes, at least 1 hour, at least 2 hours, at least 5 hours, at least 8 hours, at least 10 hours, at least 15 hours, at least 20 hours, at least 24 hours, at least 36 hours, or at least 48 hours, such as between 10 seconds and 48 hours. In an embodiment, the acetyl trifluoromethylphenyl valylglycine is not removed prior to application of one or more other skin compositions, such as, e.g., a composition comprising at least one retinoid or derivative, an additional skin peel composition, moisturizers, sunscreen, other anti-aging treatments, etc.

In some embodiments, the at least one retinoid or derivative thereof can be left on the skin for at least 10 seconds, at least 20 seconds, at least 30 seconds, at least 40 seconds, at least 50 seconds, at least 1 minute, at least 2 minutes, at least 3 minutes, at least 5 minutes, at least 10 minutes, at least 15 minutes, at least 20 minutes, at least 25 minutes, at least 30 minutes, at least 35 minutes, at least 40 minutes, at least 45 minutes, at least 1 hour, at least 2 hours, at least 5 hours, at least 8 hours, at least 10 hours, at least 15 hours, at least 20 hours, at least 24 hours, at least 36 hours, or at least 48 hours, such as between 10 seconds and 48 hours. In an embodiment, the at least one retinoid or derivative thereof is not removed prior to application of one or more other skin compositions, such as, e.g., a composition comprising acetyl trifluoromethylphenyl valylglycine, an additional skin peel composition, moisturizers, sunscreen, other anti-aging treatments, etc.

In certain embodiments, the methods comprise applying an amount of a composition comprising at least one additional chemical skin peeling agent such that about 0.1 gram or more of the at least one skin peeling agent is applied to the skin. For example, the composition may be applied such that about 0.05 g/dm² or more, such as about 0.06 g/dm² or more, such as about 0.07 g/dm² or more, such as about 0.08 g/dm² or more, such as about 0.09 g/dm² or more, such as about 0.1 g/dm² or more, such as about 0.15 g/dm² or more, about 0.2 g/dm² or more, about 0.3 g/dm² or more, about 0.4 g/dm² or more, about 0.5 g/dm² or more, about 0.75 g/dm² or more, about 1 g/dm² or more, about 1.5 g/dm² or more, about 2 g/dm² or more, about 2.5 g/dm² or more, or about 3 g/dm² or more of the at least one skin peeling agent is applied to the skin. In some embodiments, the composition is applied such that about 0.05 to about 10 g/dm², about 0.06 to about 9 g/dm², about 0.07 to about 8 g/dm², about 0.08 to about 7 g/dm², about 0.09 to about 6 g/dm², about 0.1 to about 5 g/dm², about 0.15 to about 4 g/dm², about 0.2 to about 3 g/dm², about 0.3 to about 2 g/dm², about 0.4 to about 1 g/dm²; about 0.5 to about 10 g/dm², about 0.5 to about 9 g/dm², about 0.5 to about 8 g/dm², about 0.5 to about 7 g/dm², about 0.5 to about 6 g/dm², about 0.5 to about 5 g/dm², about 0.5 to about 4 g/dm², about 0.5 to about 3 g/dm², about 0.5 to about 2 g/dm², about 0.5 to about 1 g/dm²; or about 1 to about 10 g/dm², about 1 to about 9 g/dm², about 1 to about 8 g/dm², about 1 to about 7 g/dm², about 1 to about 6 g/dm², about 1 to about 5 g/dm², about 1 to about 4 g/dm², about 1 to about 3 g/dm², or about 1 to about 2 g/dm² of the at least one additional chemical skin peeling agent is applied to the skin.

In some embodiments, the at least one additional chemical skin peeling agent can be left on the skin for at least 10 seconds, at least 20 seconds, at least 30 seconds, at least 40 seconds, at least 50 seconds, at least 1 minute, at least 2 minutes, at least 3 minutes, at least 5 minutes, at least 10 minutes, at least 15 minutes, at least 20 minutes, at least 25 minutes, at least 30 minutes, at least 35 minutes, at least 40 minutes, at least 45 minutes, at least 1 hour, at least 2 hours, at least 5 hours, at least 8 hours, at least 10 hours, at least 15 hours, at least 20 hours, at least 24 hours, at least 36 hours, or at least 48 hours, such as between 10 seconds and 48 hours, before the at least one chemical peel is removed, such as, e.g., by rinsing. In an embodiment, the at least one chemical peel is not removed prior to application of one or more other skin compositions, such as, e.g., a composition comprising acetyl trifluoromethylphenyl valylglycine, a composition comprising a retinoid or derivative thereof, moisturizers, sunscreen, other anti-aging treatments, etc.

The at least one chemical peel can be applied in any manner known. For example, it may be applied directly to the skin (e.g. with the fingers) or using an implementation such as a ball or a pad particularly adapted for surface wiping. The pad may include an absorbent core having the ability to absorb and retain liquid material, and a liner layer in contact with and covering at least one side of the absorbent core. The liner layer has the ability to retain and wick liquid material through the liner layer. The pad may be fabricated from any convenient material, where suitable materials of interest include, but are not limited to cellulosic materials, polymeric materials, etc. The wipe can be formed of a paper, cotton or textile-based substrate. In some instances, the absorbent pad material is made up of hydrophilic materials. In other embodiments, absorbent patches are selected from the group consisting of guazes, cellulosic pads, agarose gels, acrylamide gels, or hydrogels.

The ball or pad can be soaked with about 0.5 mL to about 10 mL of a composition comprising the at least one chemical peel, such as about 0.7 mL to about 9 mL, about 0.9 mL to about 8 mL, about 1 mL to about 7 mL, about 1.5 mL to about 6 mL, about 2 mL to about 5 mL, about 2 mL to about 4 mL, about 2 mL to about 3 mL, about 3 mL to about 5 mL, or about 4 mL to about 5 mL of a composition comprising the at least one chemical peel. When using a pad or ball, the at least one chemical peel can be applied by making at least 1 pass of the ball or pad over the skin, such as at least 2 passes, at least 3 passes, at least 4 passes, at least 5 passes, at least 6 passes, at least 7 passes, at least 8 passes, at least 9 passes, or at least 10 passes, such as from 1 to 10 passes, 1 to 9 passes, 1 to 8 passes, 1 to 7 passes, 1 to 6 passes, 1 to 5 passes, 1 to 4 passes, 1 to 3 passes, or from 1 to 2 passes.

The at least one pad particularly adapted for surface wiping may comprise a composition comprising at least one skin peeling agent. The wipe may also contain routine skin permeation enhancers known in the art. In an embodiment, the wipe is soaked in agents such as water, phosphate buffered saline, soap, surfactant, inorganic or organic solvents such as water, ethanol, isopropanol or a combination thereof. An exemplary formulation of the agent contains 30-95% of isopropanol in water and the wipe material is gauze. The at least one skin peeling agent is typically applied to the target skin area by massaging, wiping, padding, rubbing or any other method to clean the target skin site and to increase porosity.

When the (a) acetyl trifluoromethylphenyl valylglycine and the (b) at least one retinoid or derivative thereof are applied consecutively, the methods disclosed herein may include, in various embodiments, applying acetyl trifluoromethylphenyl valylglycine to the skin within about 24 hours before and/or after applying the at least one retinoid or derivative thereof to the skin. For example, in some embodiments the acetyl trifluoromethylphenyl valylglycine may be applied within about 20 hours, within about 16 hours, within about 12 hours, within about 10 hours, within about 8 hours, within about 6 hours, within about 4 hours, within about 2 hours, within about 1 hour, within about 30 minutes, within about 20 minutes, within about 10 minutes, within about 5 minutes, or within about 1 minute after the application of the at least one retinoid or derivative thereof to the skin. In other embodiments, the acetyl trifluoromethylphenyl valylglycine may be applied within about 20 hours, within about 16 hours, within about 12 hours, within about 10 hours, within about 8 hours, within about 6 hours, within about 4 hours, within about 2 hours, within about 1 hour, within about 30 minutes, within about 20 minutes, within about 10 minutes, within about 5 minutes, or within about 1 minute before the application of the at least one retinoid or derivative thereof to the skin.

When the (a) acetyl trifluoromethylphenyl valylglycine and (b) at least one additional skin peeling agent are applied consecutively, the methods disclosed herein may include, in various embodiments, applying acetyl trifluoromethylphenyl valylglycine to the skin within about 48 hours before or after applying the at least one skin peeling agent to the skin. For example, in some embodiments the acetyl trifluoromethylphenyl valylglycine may be applied within about 24 hours, within about 20 hours, within about 16 hours, within about 12 hours, within about 10 hours, within about 8 hours, within about 6 hours, within about 4 hours, within about 2 hours, within about 1 hour, within about 30 minutes, within about 20 minutes, within about 10 minutes, within about 5 minutes, or within about 1 minute after the application of the at least one additional skin peeling agent to the skin. In other embodiments, the acetyl trifluoromethylphenyl valylglycine may be applied within about 24 hours, within about 20 hours, within about 16 hours, within about 12 hours, within about 10 hours, within about 8 hours, within about 6 hours, within about 4 hours, within about 2 hours, within about 1 hour, within about 30 minutes, within about 20 minutes, within about 10 minutes, within about 5 minutes, or within about 1 minute before the application of the at least one additional skin peeling agent to the skin.

In some embodiments, the composition comprising acetyl trifluoromethylphenyl valylglycine can be left on the skin for at most 10 seconds, at most 20 seconds, at most 30 seconds, at most 40 seconds, at most 50 seconds, at most 1 minute, at most 2 minutes, at most 3 minutes, at most 5 minutes, at most 10 minutes, at most 15 minutes, at most 20 minutes, at most 25 minutes, at most 30 minutes, at most 35 minutes, at most 40 minutes, at most 45 minutes, at most 1 hour, at most 2 hours, at most 5 hours, at most 8 hours, at most 10 hours, at most 15 hours, at most 20 hours, at most 24 hours, at most 36 hours, or at most 48 hour before the acetyl trifluoromethylphenyl valylglycine is removed such as by, e.g., rinsing. In an embodiment, the composition comprising acetyl trifluoromethylphenyl valylglycine is not removed prior to application of other skin compositions, such as, e.g, a composition comprising a retinoid or derivative thereof, a composition comprising an additional skin peeling agent, moisturizers, sunscreen, other anti-aging treatments, etc.

The acetyl trifluoromethylphenyl valylglycine can, in various embodiments, be applied at least one time a day, at least two times a day, at least three times a day, at least four times a day, or at least five times a day, such as from one to five times a day, from one to four times a day, from one to three times a day, or from one to two times a day.

In some embodiments, the composition comprising at least one retinoid or derivative thereof is left on the skin for at least 10 seconds, at least 20 seconds, at least 30 seconds, at least 40 seconds, at least 50 seconds, at least 1 minute, at least 2 minutes, at least 3 minutes, at least 5 minutes, at least 10 minutes, at least 15 minutes, at least 20 minutes, at least 25 minutes, at least 30 minutes, at least 35 minutes, at least 40 minutes, at least 45 minutes, at least 1 hour, at least 2 hours, at least 5 hours, at least 8 hours, at least 10 hours, at least 15 hours, at least 20 hours, at least 24 hours, at least 36 hours, or at least 48 hours before it is removed, such as by, e.g., rinsing. In an embodiment, the composition comprising at least one retinoid or derivative thereof is left on the skin for at most 10 seconds, at most 20 seconds, at most 30 seconds, at most 40 seconds, at most 50 seconds, at most 1 minute, at most 2 minutes, at most 3 minutes, at most 5 minutes, at most 10 minutes, at most 15 minutes, at most 20 minutes, at most 25 minutes, at most 30 minutes, at most 35 minutes, at most 40 minutes, at most 45 minutes, at most 1 hour, at most 2 hours, at most 5 hours, at most 8 hours, at most 10 hours, at most 15 hours, at most 20 hours, at most 24 hours, at most 36 hours, or at most 48 hour before it is removed, such as by, e.g., rinsing. For example, the composition comprising at least one retinoid or derivative thereof may be left on the skin for a period of time ranging from 5 minutes to 60 minutes, such as from 5 minutes to 50 minutes, from 5 minutes to 40 minutes, from 5 minutes to 35 minutes, from 5 minutes to 30 minutes, from 5 minutes to 25 minutes, from 5 minutes to 20 minutes, from 5 minutes to 15 minutes, or from 5 minutes to 10 minutes. As a further example, the composition comprising at least one retinoid or derivative thereof may be left on the skin for a period of time ranging from 10 minutes to 60 minutes, such as from 10 minutes to 50 minutes, from 10 minutes to 40 minutes, from 10 minutes to 35 minutes, from 10 minutes to 30 minutes, from 10 minutes to 25 minutes, from 10 minutes to 20 minutes, or from 10 minutes to 15 minutes.

In some embodiments, the composition comprising at least one additional skin peeling agent is left on the skin for at least 10 seconds, at least 20 seconds, at least 30 seconds, at least 40 seconds, at least 50 seconds, at least 1 minute, at least 2 minutes, at least 3 minutes, at least 5 minutes, at least 10 minutes, at least 15 minutes, at least 20 minutes, at least 25 minutes, at least 30 minutes, at least 35 minutes, at least 40 minutes, at least 45 minutes, at least 1 hour, at least 2 hours, at least 5 hours, at least 8 hours, at least 10 hours, at least 15 hours, at least 20 hours, at least 24 hours, at least 36 hours, or at least 48 hours before it is removed, such as by, e.g., rinsing. In an embodiment, the composition comprising at least one additional skin peeling agent is left on the skin for at most 10 seconds, at most 20 seconds, at most 30 seconds, at most 40 seconds, at most 50 seconds, at most 1 minute, at most 2 minutes, at most 3 minutes, at most 5 minutes, at most 10 minutes, at most 15 minutes, at most 20 minutes, at most 25 minutes, at most 30 minutes, at most 35 minutes, at most 40 minutes, at most 45 minutes, at most 1 hour, at most 2 hours, at most 5 hours, at most 8 hours, at most 10 hours, at most 15 hours, at most 20 hours, at most 24 hours, at most 36 hours, or at most 48 hours. For example, compositions comprising the at least one additional skin peeling agent may be left on the skin for a period of time ranging from 5 minutes to 60 minutes, such as from 5 minutes to 50 minutes, from 5 minutes to 40 minutes, from 5 minutes to 35 minutes, from 5 minutes to 30 minutes, from 5 minutes to 25 minutes, from 5 minutes to 20 minutes, from 5 minutes to 15 minutes, or from 5 minutes to 10 minutes. As a further example, the composition comprising at least one additional skin peeling agent may be left on the skin for a period of time ranging from 10 minutes to 60 minutes, such as from 10 minutes to 50 minutes, from 10 minutes to 40 minutes, from 10 minutes to 35 minutes, from 10 minutes to 30 minutes, from 10 minutes to 25 minutes, from 10 minutes to 20 minutes, or from 10 minutes to 15 minutes.

The methods may, in some embodiments, include applying a composition comprising acetyl trifluoromethylphenyl valylglycine to the skin more than one time, e.g. before and after the application of the at least one retinol and/or the at least one skin peeling agent to the skin. The methods may, in some embodiments, include applying the at least one retinol to the skin more than one time, e.g. before and after the application of the acetyl trifluoromethylphenyl valylglycine and/or the at least one chemical peel to the skin. In some cases, the method may be repeated to achieve optimal results. The methods may, in some embodiments, include applying the at least one skin peeling agent to the skin more than one time, e.g. before and after the application of the acetyl trifluoromethylphenyl valylglycine and/or the at least one retinol to the skin. In some cases, the method may be repeated to achieve optimal results.

If applied simultaneously, the acetyl trifluoromethylphenyl valylglycine and the retinoid(s) or derivative(s) may optionally be present in a single composition. Alternatively, the acetyl trifluoromethylphenyl valylglycine and the retinoid(s) or derivative(s) may optionally be present in separate compositions, e.g. two or more compositions, three or more compositions, etc., which may be mixed at or near the time of use.

If applied simultaneously, the acetyl trifluoromethylphenyl valylglycine and the skin peeling agent(s) may optionally be present in a single composition. Alternatively, the acetyl trifluoromethylphenyl valylglycine and the skin peeling agent(s) may optionally be present in separate compositions, e.g. two or more compositions, three or more compositions, etc., which may be mixed at or near the time of use.

The skin treatment compositions may be used in methods for increasing or improving skin renewal/turnover, and/or improving the appearance of skin. Non-limiting examples of such methods include methods of treating fine lines, methods of treating under eye wrinkles, and/or methods of treating nasolabial folds.

In some exemplary embodiments, the acetyl trifluoromethylphenyl valylglycine can be applied to the skin at least one time per day, for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, or 84 consecutive or non-consecutive days, preferably consecutive days. The acetyl trifluoromethylphenyl valylglycine may be applied to the skin at least one time per day, at least 1, 2, 3, 4, 5, 6, or 7 days per week, preferably at least 2 times per day. For example, the acetyl trifluoromethylphenyl valylglycine may be applied to the skin at least two times per day, wherein the second application is at least about 6 hours, preferably at least about 8 hours, more preferably at least about 10 hours, most preferably at least about 12 hours after the first application.

In some exemplary embodiments, the at least one retinoid or derivative thereof can be applied to the skin at least one time per day for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 consecutive or non-consecutive days, preferably nonconsecutive days. The retinoid may be applied to the skin at least one time per day at least 1, 2, 3, 4, 5, 6, or 7 times per week. For example, the at least one retinoid or derivative thereof can be applied to the skin at least every other day.

In some exemplary embodiments, the at least one additional skin peeling agent can be applied to the skin at least one time per day for at least 1, 2, 3, 4, 5, 6, 7, 8, 9, 10, 11, 12, 13, 14, 15, 16, 17, 18, 19, 20, 21, 22, 23, 24, 25, 26, 27, or 28 consecutive or non-consecutive days, preferably nonconsecutive days. The at least one additional skin peeling agent may be applied to the skin at least one time per day at least 1, 2, 3, 4, 5, 6, or 7 days a week. For example, the at least one additional skin peeling agent can be applied to the skin at least every other day.

In some exemplary embodiments, the additional skin peeling agent may be applied to the skin at least every other day for a period of at least 1, 2, 3, 4, 5, 6, 7, 8, 9, 10, 11, 12, 13, 14, 15, 16, 17, 18, 19, 20, 21, 22, 23, 24, 25, 26, 27, or 28 days, followed by a period of at least 1, 2, 3, 4, 5, 6, 7, 8, 9, 10, 11, 12, 13, 14 days wherein the additional skin peeling agent is not applied.

In some embodiments, the at least one retinoid or derivative thereof and the at least one additional skin peeling agent are not applied to skin on the same day.

In various embodiments, the methods comprise two phases for treating skin, comprising:
a phase (1) comprising applying to the skin:
   (a) acetyl trifluoromethylphenyl valylglycine at least one time per day;
   (b) at least one retinoid or derivative thereof at least one time every two to three days; and
   (c) optionally at least one additional skin peeling agent at least one time every two to three days,
wherein the (b) at least one retinoid or derivative thereof and the (c) optional at least one additional skin peeling agent are separately applied to the skin at least about 6 hours, preferably at least about 12 hours, more preferably at least about 18 hours, most preferably at least about 24 hours apart; and
wherein phase (1) lasts for a period of time of at least about 10 days, preferably at least about 14 days, more preferably at least about 21 days, most preferably at least about 28 days; and
a phase (2) comprising applying to the skin:
   (a) acetyl trifluoromethylphenyl valylglycine at least one time per day; and
   (b) at least one retinoid or derivative thereof at least one time every two to three days; and
wherein phase (2) lasts for a period of time of at least about 5 days, preferably at least about 7 days, more preferably at least about 10 days, most preferably at least about 14 days.

In further embodiments, the methods comprise two phases for treating skin, comprising:
a phase (1) comprising separately applying to the skin:
   (a) a composition comprising acetyl trifluoromethylphenyl valylglycine, at least one time per day;
   (b) a composition comprising at least one retinoid or derivative thereof, at least one time every two to three days; and

(c) optionally a composition comprising at least one additional skin peeling agent, at least one time every two to three days,
wherein the (b) composition comprising at least one retinoid or derivative thereof and the (c) optional composition comprising at least one additional skin peeling agent are applied to the skin at least about 6 hours, preferably at least about 12 hours, more preferably at least about 18 hours, most preferably at least about 24 hours apart; and
wherein phase (1) lasts for a period of time of at least about 10 days, preferably at least about 14 days, more preferably at least about 21 days, most preferably at least about 28 days; and
a phase (2) comprising separately applying to the skin:
   (a) a composition comprising acetyl trifluoromethylphenyl valylglycine, at least one time per day; and
   (b) a composition comprising at least one retinoid or derivative thereof, at least one time every two to three days; and
wherein phase (2) lasts for a period of time of at least about 5 days, preferably at least about 7 days, more preferably at least about 10 days, most preferably at least about 14 days.

In yet further embodiments, the methods comprise two phases for treating skin, comprising:
a phase (1) comprising separately applying to the skin:
   (a) a composition comprising acetyl trifluoromethylphenyl valylglycine, at least one time per day;
   (b) a composition comprising at least one retinoid or derivative thereof and acetyl trifluoromethylphenyl valylglycine, at least one time every two to three days; and
   (c) optionally a composition comprising at least one additional skin peeling agent, at least one time every two to three days,
wherein the (b) composition comprising at least one retinoid or derivative thereof and acetyl trifluoromethylphenyl valylglycine and the (c) optional composition comprising at least one additional skin peeling agent are applied to the skin at least about 6 hours, preferably at least about 12 hours, more preferably at least about 18 hours, most preferably at least about 24 hours apart; and
wherein phase (1) lasts for a period of time of at least about 10 days, preferably at least about 14 days, more preferably at least about 21 days, most preferably at least about 28 days; and
a phase (2) comprising separately applying to the skin:
   (a) a composition comprising acetyl trifluoromethylphenyl valylglycine, at least one time per day; and
   (b) a composition comprising at least one retinoid or derivative thereof and acetyl trifluoromethylphenyl valylglycine, at least one time every two to three days; and
wherein phase (2) lasts for a period of time of at least about 5 days, preferably at least about 7 days, more preferably at least about 10 days, most preferably at least about 14 days.

In still further embodiments, the methods comprise two phases for treating skin, comprising:
a phase (1) comprising applying to the skin:
   (a) from about 0.1 to about 10 wt % of acetyl trifluoromethylphenyl valylglycine, at least one time, preferably at least two times, per day;
   (b) from about 0.001 to about 10 wt % of at least one retinoid or derivative thereof, at least one time every two to three days; and
   (c) optionally from about 0.1 to about 25 wt % of at least one additional skin peeling agent chosen from α-hydroxy acids, β-hydroxy acids, or mixtures thereof, at least one time every two to three days,
wherein the (b) at least one retinoid or derivative thereof and the (c) optional at least one additional skin peeling agent are separately applied to the skin at least about 6 hours, preferably at least about 12 hours, more preferably at least about 18 hours, most preferably at least about 24 hours apart; and
wherein phase (1) lasts for a period of time of at least about 10, preferably at least about 14 days, more preferably at least about 21 days, most preferably at least about 28 days; and
a phase (2) comprising applying to the skin:
   (a) from about 0.1 to about 10 wt % of acetyl trifluoromethylphenyl valylglycine, at least one time, preferably at least two times, per day; and
   (b) from about 0.001 to about 10 wt % of at least one retinoid or derivative thereof, at least one time every two to three days; and
wherein phase (2) lasts for a period of time of at least about 5, preferably at least about 7, more preferably at least about 10, most preferably at least about 14 days;

wherein amounts of all components are relative to the total weight of the composition in which it is present.

It should be understood that in any method according to the disclosure, including the exemplary methods described above, methods comprising more than one phase, e.g. a two-phase method, may differ in any way, without limitation. For example, in one embodiment, a two-phase method may differ only in the presence of a (c) skin peeling agent other than retinoids or derivatives thereof, e.g. where phase (1) includes application of (c) but phase (2) does not. As a further example, in one embodiment, a two-phase method may differ in the concentration of one or more components applied to the skin during the various phases, and/or in the length of time phase (1) or phase (2) lasts. For example, phase (1) and phase (2) may use (a) compositions comprising identical concentrations of the amount of acetyl trifluoromethylphenyl valylglycine in each phase, but (b) compositions comprising the same retinoid or derivative thereof that are present in the compositions in different concentrations in each phase. As another example, phase (1) and phase (2) may use (a) compositions comprising identical concentrations of the amount of acetyl trifluoromethylphenyl valylglycine in each phase, but (b) compositions comprising different retinoids or derivatives thereof, present in the compositions in the same or different concentrations in each phase. As a still further example, phase (1) and phase (2) may use (a) compositions comprising different concentrations of the amount of acetyl trifluoromethylphenyl valylglycine in each phase, but (b) compositions comprising the same retinoid or derivative thereof that are present in the same concentration in each phase, and so on.

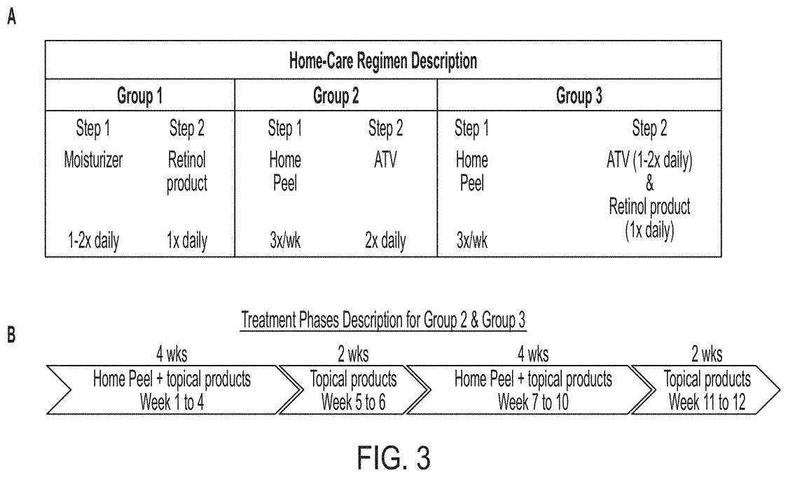

It is also to be understood that, in multiple-phase methods according to the disclosure including the exemplary methods described above, the order and timing of each phase is not limited. By way of non-limiting example, as shown in FIG. 3B, one phase may follow another substantially sequentially in time, e.g. where phase (2) begins approximately as soon as phase (1) ends (with or without a rest period) or vice versa. In addition, as also shown in FIG. 3B, a multi-phase method may be repeated as desired, e.g. a phase (2) may begin approximately as soon as a first phase (1) ends, and a second phase (1) may begin approximately as soon as the phase (2) ends, and so on as desired.

It has also been surprisingly and unexpectedly discovered that the use of acetyl trifluoromethylphenyl valylglycine reduces skin irritation, redness, etc. typically observed with the use of retinoids and derivatives thereof. Therefore, in further embodiments, the methods relate to improving skin tolerance to retinoids and/or derivatives thereof, comprising applying acetyl trifluoromethylphenyl valylglycine and at least one retinoid or derivative thereof simultaneously or sequentially, for example as described for any of the various embodiments above.

### III. Kits

The disclosure also relates to kits comprising acetyl trifluoromethylphenyl valylglycine, at least one retinoid or derivative thereof, and optionally at least one skin peeling agent. In further embodiments, the kits comprise acetyl trifluoromethylphenyl valylglycine, at least one retinoid or derivative thereof, and/or at least one exfoliating implementation, such as, for example, a pad for exfoliating the skin which may or may not comprise a chemical exfoliating agent.

In some embodiments, the kits comprise at least two or three separate containers or compartments. In further embodiments, the kits comprise one or more containers, wherein at least one container comprises at least two separate compartments.

In some embodiments, kits comprise a first container or compartment that comprises (a) a composition comprising acetyl trifluoromethylphenyl valylglycine, a second container or compartment that comprises (b) a composition comprising at least one retinoid or derivative thereof, and optionally a third container or compartment that comprises (c) a composition comprising at least one additional skin peeling agent. In further embodiments, kits comprise a first container or compartment that comprises (a) a composition comprising acetyl trifluoromethylphenyl valylglycine and (b) a composition comprising at least one retinoid or derivative thereof, and optionally a second container or compartment that comprises (c) a composition comprising at least one additional skin peeling agent. The kits may optionally further comprise at least one additional container or compartment.

By way of non-limiting example only, the kits may be in the form of a dual- or triple-chamber container. The first, second, and/or third chambers may be separate such that compositions (a), (b), and/or (c) are segregated from each other prior to use. The dual-chamber container may optionally comprise an implementation for mixing and/or dispensing amounts of compositions (a), (b), and/or (c) such that compositions (a), (b) and/or (c) are dispensed as a single composition comprising the desired amounts of acetyl trifluoromethylphenyl valylglycine, retinoid(s) or derivatives thereof, and/or additional skin peeling agent(s) for use in various methods according to the disclosure.

As a further non-limiting example, the kits may comprise a first container or compartment with a dispenser, wherein the first container or compartment comprises (a) a composition comprising acetyl trifluoromethylphenyl valylglycine, a second container or compartment with a dispenser, wherein the second container or compartment comprises (b) a composition comprising at least retinoid or derivative thereof, and/or a third container or compartment with a dispenser, wherein the third container or compartment comprises (c) a composition comprising at least one additional skin peeling agent. Optionally, the kits may further comprise instructions providing dosage, order, and timing of application, etc., of compositions (a) and (b); (a)/(b) and (c); (a), (b), and (c); etc., for use in various methods according to the disclosure. Kits according to the disclosure may also comprise an implementation for exfoliating the skin in addition to or instead of a composition comprising a chemical peeling agent, e.g. a pad for exfoliating the skin which may or may not comprise a chemical exfoliating agent.

In certain embodiments, kits according to the disclosure include a first container or compartment comprising a composition comprising acetyl trifluoromethylphenyl valylglycine and at least one retinoid or derivative thereof, and optionally a second container or compartment comprising at least one additional skin peeling agent or at least one exfoliating implementation. In other embodiments, kits according to the disclosure include a first container or compartment comprising a composition comprising acetyl trifluoromethylphenyl valylglycine, a second container or compartment comprising at least one retinoid or derivative thereof, and optionally a third container or compartment comprising at least one additional skin peeling agent or at least one exfoliating implementation.

In other embodiments, kits according to the disclosure include a first container or compartment comprising a composition comprising acetyl trifluoromethylphenyl valylglycine and at least one additional peeling agent, and optionally a second container or compartment comprising at least one retinoid or derivative thereof.

In further embodiments, kits according to the disclosure include a first container or compartment comprising (a) compositions comprising acetyl trifluoromethylphenyl valylglycine that are free or substantially free of retinoids or derivatives thereof and/or additional skin peeling agents; a second container or compartment comprising (b) compositions comprising retinoids or derivatives thereof that are free or substantially free of acetyl trifluoromethylphenyl valylglycine and/or additional skin peeling agents; and/or a third container or compartment comprising (c) compositions comprising one or more skin peeling agent(s) that are free or substantially free of acetyl trifluoromethylphenyl valylglycine and/or retinoids or derivatives thereof. In still further embodiments, kits according to the disclosure include a first container or compartment comprising (a) compositions comprising acetyl trifluoromethylphenyl valylglycine that are free or substantially free of retinoids or derivatives thereof and/or additional skin peeling agents; a second container or compartment comprising (b) compositions comprising retinoids or derivatives thereof that are free or substantially free of acetyl trifluoromethylphenyl valylglycine and/or additional skin peeling agents; and/or at least one exfoliating implementation.

Having described various embodiments of the present disclosure in detail, it will be apparent that modifications and variations thereof are possible without departing from the scope of the disclosure. Furthermore, it should be appreciated that all examples in the present disclosure, while illustrating embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof. Thus, where the disclosure refers to an element “chosen from A, B, C, D, E, F, and/or mixtures thereof,” it indicates that that one of A, B, C, D, and F may be included (e.g. only A, only B, only C, etc.), or a mixture of any two of A, B, C, D, E, and F may be included (e.g. a mixture of A and B, a mixture of A and C, a mixture of C, D, and E, etc.).

As used herein, the phrases “and mixtures thereof,” “and a mixture thereof,” “and combinations thereof,” “and a combination thereof,” “or mixtures thereof,” “or a mixture thereof,” “or combinations thereof,” “or a combination thereof,” and variations thereof are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

Unless otherwise indicated, all percentages herein are by weight, relative to the total weight of the composition.

All percentages, parts, and ratios herein are based upon the total weight of the compositions of the present disclosure, unless otherwise indicated.

For purposes of the disclosure, in multiple-phase treatment methods, amounts of components used in each phase are independently chosen, i.e. the amounts of components may be the same as, or may be different from, the amount of the corresponding component in another phase. For example, in a two-phase method of treating skin with (a) compositions comprising acetyl trifluoromethylphenyl valylglycine in both phases, the amount of acetyl trifluoromethylphenyl valylglycine in composition (a) of phase (1) may or may not be the same as the amount of acetyl trifluoromethylphenyl valylglycine in composition (a) of phase (2).

As used herein, the term “treat” (and its grammatical variations, such as “treating”) refers to the application of the compositions of the present disclosure onto the surface of the skin. By “treating” the skin according to the disclosure, the appearance of various signs of aging such as such fine lines, under eye wrinkles, and/or nasolabial folds is diminished.

As used herein, the terms “skin turnover,” “epidermal turnover,” “skin renewal” and “epidermal renewal” are used interchangeably to refer to the skin's process for turning over skin cells, including the formation of new cells and/or the shedding of old cells.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, the term “substantially free” or “essentially free” as used herein means the specific material may be present in small amounts that do not materially affect the basic and novel characteristics of the compositions according to the disclosure. For instance, there may be less than 2 wt % by weight of a specific material added to a composition, based on the total weight of the compositions (provided that an amount of less than 2 wt % by weight does not materially affect the basic and novel characteristics of the compositions according to the disclosure. Similarly, the compositions may include less than 2 wt %, less than 1.5 wt %, less than 1 wt %, less than 0.5 wt %, less than 0.1 wt %, less than 0.05 wt %, or less than 0.01 wt %, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1 wt % to 10 wt %, such as 2 wt % to 8 wt %, such as 3 wt % to 5 wt %,” is intended to encompass ranges of “1 wt % to 8 wt %,” “1 wt % to 5 wt %,” “2 wt % to 10 wt %,” and so on. All numbers, amounts, ranges, etc., are intended to be modified by the term “about,” whether or not so expressly stated. Similarly, a range given of “about 1 wt % to 10 wt %” is intended to have the term “about” modifying both the 1 wt % and the 10 wt % endpoints. The term “about” is used herein to indicate a difference of up to +/−10 wt % from the stated number, such as +/−9 wt %, +/−8 wt %, +/−7 wt %, +/−6 wt %, +/−5 wt %, +/−4 wt %, +/−3 wt %, +/−2 wt %, or +/−1 wt %. Likewise, all endpoints of ranges are understood to be individually disclosed, such that, for example, a range of 1:2 to 2:1 is understood to disclose a ratio of both 1:2 and 2:1.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples serve to illustrate the embodiments of the disclosure without however exhibiting a limiting character. The Examples are intended to be non-restrictive and explanatory only, with the scope of the invention being defined by the claims. In the examples the amounts of the composition ingredients are given as weight percentages of active ingredients relative to the total weight of the composition.

### Example 1—Effect on Skin Turnover

The effect of acetyl trifluoromethylphenyl valylglycine and/or retinoids on skin turnover/desquamation, filaggrin expression, desmocollin-1 expression was evaluated in an ex vivo skin model. In this study, the effects of acetyl trifluoromethylphenyl valylglycine (0.1%), retinol (0.003%) and acetyl trifluoromethylphenyl valylglycine+retinol on the skin were evaluated by H&E (assessing for overall tissue health), filaggrin (assessing the level of barrier renewal) and desmocollin-1 (assessing the tight junction between the keratinocytes).

Fresh post-abdominoplasty normal human skin samples (3 lots, age 36-59 years old, Hispanic/Caucasian/African American, female) were acquired from BioIVT Inc. (Westbury, N.Y.). Tissue was defatted and cleaned of blood residue. Explant samples were then created using a 12 mm biopsy punch. Samples were cleaned with sterile PBS. Skin biopsies not receiving treatment served as controls. Skin explants were maintained at air-liquid interface at 37° C. and 5% CO2 in four different treatment groups (n=3 per media type):
1. Dulbecco's Modified Eagle's Medium (DMEM) with 10% Fetal Bovine Serum and 1% Penicillin-Streptomycin, and 2% ethanol, serving as the vehicle control;
2. Dulbecco's Modified Eagle's Medium (DMEM) with 10% Fetal Bovine Serum, 1% Penicillin-Streptomycin, and 0.1% ER2947 in 2% ethanol, serving as the acetyl trifluoromethylphenyl valylglycine treatment group;
3. Dulbecco's Modified Eagle's Medium (DMEM) with 10% Fetal Bovine Serum, 1% Penicillin-Streptomycin, and 0.003% Retinol in 2% ethanol, serving as the Retinol treatment group; or
4. Dulbecco's Modified Eagle's Medium (DMEM) with 10% Fetal Bovine Serum, 1% Penicillin-Streptomycin, and 0.1% ER2947+0.003% retinol in 2% ethanol, serving as the acetyl trifluoromethylphenyl valylglycine/Retinol treatment group.

Fresh media was replenished every other day except for weekends. Following a 7-day culture period, samples of all explants were taken for histological and immunohistochemical analysis.

Skin explants were processed for haematoxylin and eosin, and immunohistochemical staining (Reveal Biosciences, CA). H&E staining was performed using the standard protocol (Reveal Biosciences, CA). Immunohistochemical staining was performed using a Leica Bond automated immunostainer (Reveal Biosciences, CA). Tissue samples were embedded as formalin-fixed paraffin embedded (FFPE) blocks. Heat induced antigen retrieval was performed and non-specific antibody binding was blocked. Sections were incubated with the primary antibodies Mouse Monoclonal to Anti-Filaggrin (MA5-13441, Thermofisher, MA), Rabbit Polyclonal to Anti-Kallikrein 5 (LS-B15516-50, LS Bio, WA), or Rabbit Polyclonal to Anti-Desmocollin 1 (HPA075379, Sigma, MO). The primary staining was followed by rabbit anti-mouse IgG or anti-rabbit Poly-HRP-IgG respectively and were detected using the Bond Polymer Refine Detection kit (DS9800, Leica Biosystems, UK). All sections were then counterstained with a hematoxylin nuclear stain and imaged with an inverted microscope (Leica DM500, Germany).

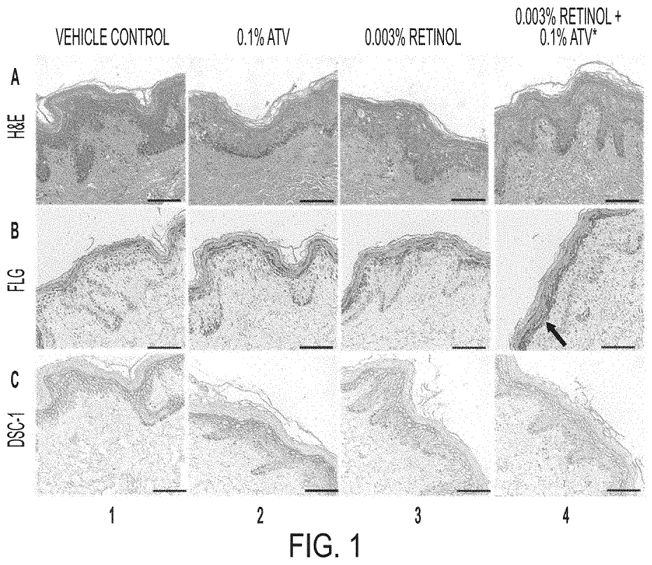

The complementary and synergistic effects of pairing acetyl trifluoromethylphenyl valylglycine with retinol are illustrated by the immunohistochemical evaluation shown in FIG. 1. FIG. 1 discloses the images of the stained explants on the seventh day of culture following application of acetyl trifluoromethylphenyl valylglycine and/or retinol. Tissues treated with 2 wt % ethanol in media in the figures serve as negative controls which demonstrate what untreated 2D ex-vivo histological cross-sections with no significant change to tissue health or evaluated biomarkers would look like. Tissue structure and integrity following retinol and/or acetyl trifluoromethylphenyl valylglycine treatment was observed amongst the hematoxylin and eosin stained and immunohistochemical stained tissue. Increases in filaggrin (FLG) expression and a reduction in desmocollin-1 (DSC1) expression were indicators of epidermal renewal. The effects of retinol can be observed by the change in H&E (illustrating some mild level of skin irritation) and downward expression of desmocollin-1 (suggesting weakening in skin barrier). The addition of acetyl trifluoromethylphenyl valylglycine increases the expression of filaggrin. This suggests that acetyl trifluoromethylphenyl valylglycine can complement the effects of retinol by inducing in the recovery and renewal of skin barrier.

### Example 2—Penetration into Skin

Human skin samples were obtained from abdominal surgery. Excess subcutaneous fat was removed, if necessary. Skin samples were excised and cut into pieces of around 2 cm×2 cm. Thickness was measured and transepidermal water loss (TEWL) was measured. The human skin was included in the study if the TEWL was between 0.5 and 13 g/m²/h for abdominal skin. The 2 cm² skin samples were kept in a static cell in a diffusion chamber in a Franz cell horizontal cell clamp system (Crown Glass Company, Inc.) and maintained at a constant temperature of 32° C.±1° C. The static cells consist of a donor chamber and a receptor chamber with a 7 mL receptor fluid compartment separated by the skin.

The cells were placed on a magnetic stirrer which was integrated with a fixed temperature water bath maintained in order to have 32° C.±1° C. at the skin surface. The receptor compartment of each cell was filled with the receptor fluid (PBS) and was continuously stirred. The skin samples were placed on the receptor compartment. The donor compartment was then placed onto the skin samples. After confirming the absence of air-bubbles, a clamp was placed to link both compartments. Agitation of the receptor fluid was started.

A composition containing 2 wt % acetyl trifluoromethylphenyl valylglycine was applied to the skin sample at a rate of 10 mg/cell using a positive displacement pipette and the exact amount applied was determined by weight then recorded (Sartorius Entris II Essential Analytical balance, Thermo-Fisher).

The experiment started immediately after the application of the preparation on the skin surface and ended 16 hours after application. The preparation was washed off the skin samples 16 hours after application.

The skin was then stripped with consistent weight and time of application. A maximum of 20 strips was performed on each skin sample and the strips were pooled by 5. The strips samples were stored with solvent (methanol) at −20° C. for a maximum of 5 days before being analyzed. Using the scalpel blade, the epidermis (E) and dermis (D) corresponding to the application area were cut and then separated by heating. Epidermis (E) and dermis (D) were weighed. The remaining skin (RS) was cut in four parts. The RS vials were stored with solvent (methanol) at −20° C. for a maximum of 7 days before being analyzed. Dermis and epidermis samples were stored with solvent at −20° C. for a maximum of 7 days before being analyzed. The receptor fluid was taken 16 hours after the application and the samples were analyzed.

Receptor fluid samples were stored at −20° C. for a maximum of 6 days before being analyzed. The donor compartment, the upper seal and the scalpel blade (LM) were placed in a flask with methanol and shaken for 2 hours. Samples were stored at −20° C. for a maximum of 5 days before being analyzed.

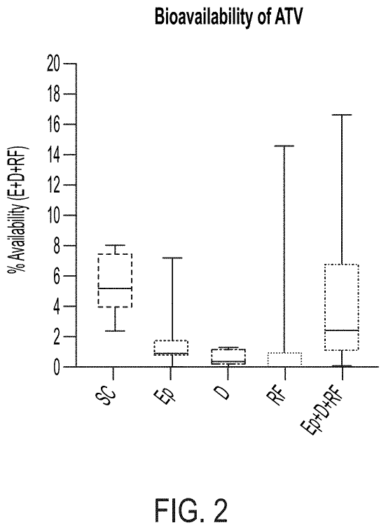

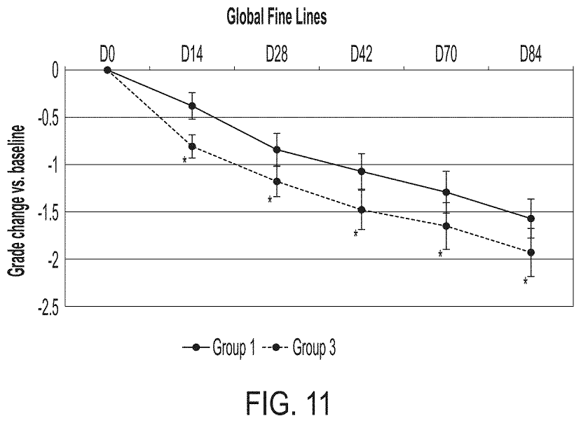

The final extract was analyzed by LC-MS/MS. Results are provided in FIG. 2. The skin penetration level (EP+D+RF in FIG. 11) of acetyl trifluoromethylphenyl valylglycine was calculated to be about 4 wt % of the 2 wt % acetyl trifluoromethylphenyl valylglycine preparation. Thus, about 0.08 wt % acetyl trifluoromethylphenyl valylglycine penetrated through the stratum corneum. As such, the dermal dose of the 2 wt % acetyl trifluoromethylphenyl valylglycine preparation based on in vitro functionality and penetration level was calculated to be at least about 0.08 wt %.

Thus, the application of the 2% acetyl trifluoromethylphenyl valylglycine composition to skin resulted in about 0.8% penetrating past the stratum corneum barrier and into the fractions of epidermis, dermis, and receptor fluid. The level of penetrated acetyl trifluoromethylphenyl valylglycine correlates to the approximate level of the dosed acetyl trifluoromethylphenyl valylglycine in the ex vivo skin (Example 1) experiments above.

### Example 3—Clinical Studies

A monocentric, full-face, randomized, and double-blinded study was conducted to evaluate the clinical efficacy and consumer perception of three home-care regimens on improving facial dyschromia and aging signs in Caucasian healthy women. One hundred and twenty-six (126) overall healthy Caucasian women, aged 40-65 years (mean age 57.5 years), presenting with mild to moderate sign of aging with Fitzpatrick skin phototype II-Ill were included in this study. Pregnant or lactating women who have used retinol or related product and/or received treatment for chemical peel, laser, photo facials, dermabrasion, botulinum toxin, injectable fillers, or other skin tightening treatments within 6 months of the study start date were excluded. Once enrolled, subjects were asked to stop their regular skin care regime. The subjects in Table 1 were trained on self-application of all test products prior to study start.

TABLE 1
Demographics of Enrolled Subjects: N = 126 (per-protocol population)

| | Group 2: | Group 3: |
|---|---|---|
| Group 1: | Home Peel & Peptide | Home Peel & Peptide + |
| Retinol product | product | Retinol product |
| N = 46 | N = 40 | N = 40 |
| Age mean (years): 58.6 | Age mean (years): 57.1 | Age mean (years): 56.7 |
| 17 Fitz II & 29 Fitz III | 19 Fitz II & 21 Fitz III | 17 Fitz II & 23 Fitz III |
| Retinol naïve users: 38; | Retinol naïve users: 33; | Retinol naïve users: 38; |
| Retinol users: 8 | Retinol users: 7 | Retinol users: 2 |

The study consisted of a 7-day washout period, followed by 12 weeks of treatment. Subjects were provided with auxiliary products (cleanser, moisturizer, and sunscreen with SPF 30) to use during the washout period. Cleanser and sunscreen were also used during the 12 weeks treatment. Following the washout period, subjects were randomized into three home-care regimen groups, as illustrated in FIG. 3A. The regimens are described below:

Regimen 1 consisted of a moisturizer, applied every morning; a retinol-containing product, applied every third night during the first week post-washout period, follow by every two days during the second week, then applied nightly for the remained of the study duration.

Regimen 2 consisted of a 2% acetyl trifluoromethylphenyl valylglycine composition, applied daily morning and evening; a 0.45% salicylic acid/11.11% lactic acid at home chemical peel applied three times a week in the evening.

Regimen 3 consisted of 2% acetyl trifluoromethylphenyl valylglycine applied daily morning and evening; a 0.45% salicylic acid/11.11% lactic acid at home chemical peel applied three times a week in the evening. Additionally, subjects were instructed to apply a combination 1.7% acetyl trifluoromethylphenyl valylglycine/3% retinol containing product twice a week on days of no chemical peel treatment during the first week post-washout period, follow by three times a week on days of no chemical peel treatment for the remaining of study duration.

Regimen Group 1 used the same product routine throughout the 12 weeks of treatment. For Regimen Groups 2 and 3, the 12-week treatment periods were divided into two phases, completed twice (FIG. 3B): 4 weeks of using the home chemical peel in combination with all of the topical products (Week 1 to Week 4 and Week 7 to Week 10), followed by 2 weeks of using the topical products, but not the chemical peel treatment (Week 5 to Week 6 and Week 11 to Week 12).

The subjects were evaluated for clinical efficacy and tolerability at baseline and weeks 2, 4, 6, 10, and 12. Photoaging parameters were assessed globally using a modified Griffith's 10-point scale according to the following numeric definition (half-point scores were used as necessary for accuracy): 0=none, 1-3=mild, 4-6=moderate, and 7-9=severe. Crow's feet and nasolabial fold wrinkles were assessed globally using the Caucasian Skin Aging Atlas volume 1 scales, ranging from 0-5 (score of 0.4 and 0.6 were used as necessary for accuracy). Tolerability was objectively assessed at each visit by investigator by evaluating signs of erythema, dryness, scaling, and peeling. In addition, subjects self-report of the degree of burning, stinging, itching, dryness, tightness, or tingling of facial skin are also recorded. Both clinical grading and subjective tolerability assessments were measured using a 3-point scale (half-point scores were used as necessary for accuracy): where 0=none; 1=mild; 2=moderate; and 3=severe.

Self-assessment questionnaires were given to subjects to evaluate their perception on facial skin condition, product efficacy and sensory at baseline, weeks 4, 6 and 12. according to the following system: agree completely, agree somewhat, neither agree nor disagree, disagree somewhat, and disagree completely.

For clinical assessment of efficacy and tolerance parameters, a frequentist and bayesian linear mixed model were used to analyze the mean difference in change from baseline with baseline, treatment, time and treatment-time interaction as fixed effects and subject as random effect to obtain differences in treatment effects and time effect. Effect size and p-values were both used to interpret significant differences (P-values<0.05 were considered statistically significant and classes of effect size).

### Tolerability Results

Table 2 below discloses mean Scores on 0-3 scale (0=none, 1=mild, 2=moderate, 3=severe).

TABLE 2
Skin Tolerance Grading (Mean score) across Home-Care Regimen Groups

| | | Week 6 | | | | Week 12 | | |
|---|---|---|---|---|---|---|---|---|
| | | Group 1 | | Group 2 | Group 3 | Group 1 | Group 2 | Group 3 |
| | | (n = 32 | | (n = 15 | (n = 15 | (n = 16 | (n = 9 | (n = 8 |
| | Parameters | reactions) | | reactions) | reactions) | reactions) | reactions) | reactions) |
| Objective | Eythema | 1 | (n = 25) | 1 (n = 12) | 1.1 (n = 12) | 1 (n = 15) | 1.2 | 1.2 |
| Assessment | Peeling | 1.3 | (n = 4) | 1 (n = 1) | 1.3 (n = 3) | 1 (n = 1) | 0 | 0 |
| (Investigator) | Dryness | 1.5 | (n = 2) | 1 (n = 1) | 0 | 0 | 0 | 0 |
| | Edema | 1.5 | (n = 1) | 0 | 0 | 0 | 0 | 0 |
| Subjective | Stinging/ | 1 | (n = 1) | 0 | 0 | 0 | 0 | 0 |
| Assessment | Burning/ | | | | | | | |
| (Subject | Itching | | | | | | | |

| | | | | | | | |
|---|---|---|---|---|---|---|---|
| reported) | Tingling | 0 | 0 | 0 | 0 | 0 | 0 |
| | Tightness | 0 | 0 | 0 | 0 | 0 | 0 |

Group 1: Retinol Product
Group 2: Home Peel & Peptide product
Group 3: Home Peel & (Peptide + Retinol product)

More subjects in in Group 1 as compared to Groups 2 and 3 experienced mild to moderate local intolerances at Week 6, with erythema signs as the driving factor. One subject also self-reported stinging, burning, and itching. By Week 12, only mild erythema reactions were clinically observed. Approximately half as many subjects in Groups 2 and 3 experienced mild to moderate local intolerances at Week 12, as compared to Group 1.

### Adverse Events

Overall, 8 subjects experienced a total of 8 non-serious adverse events (AEs) that were considered un-related to product regimen, and all resolved without sequelae. These subjects were withdrawn from the study by the investigator.

### Efficacy Evaluations

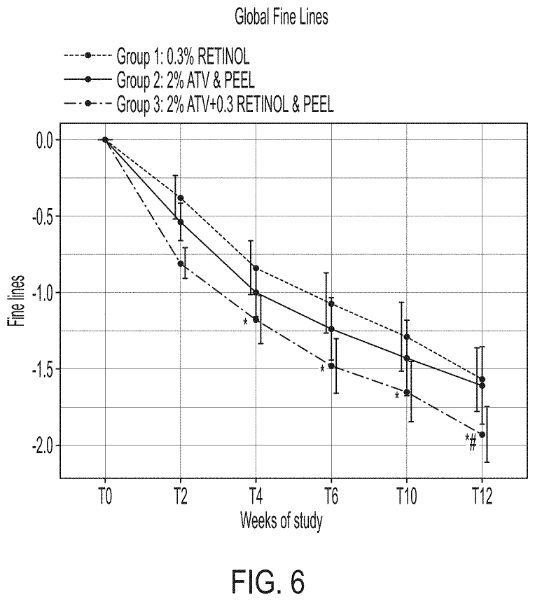

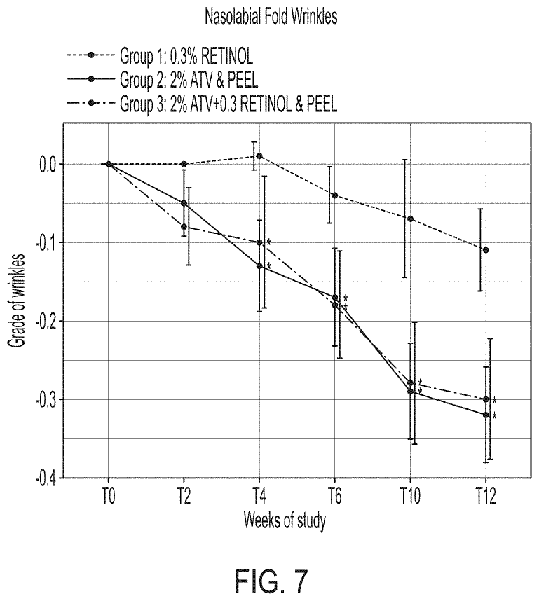

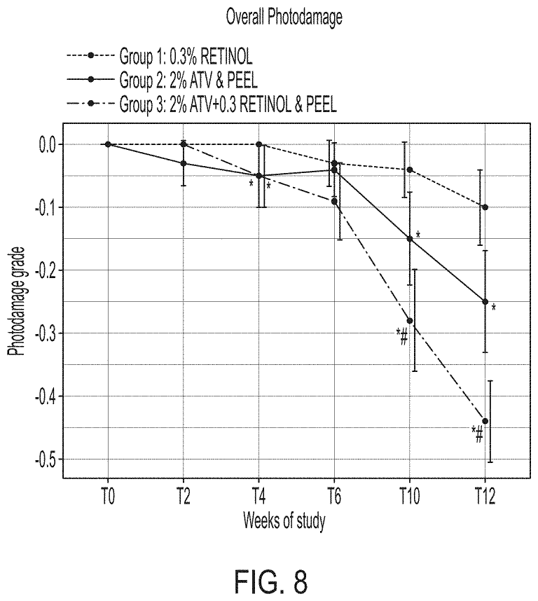

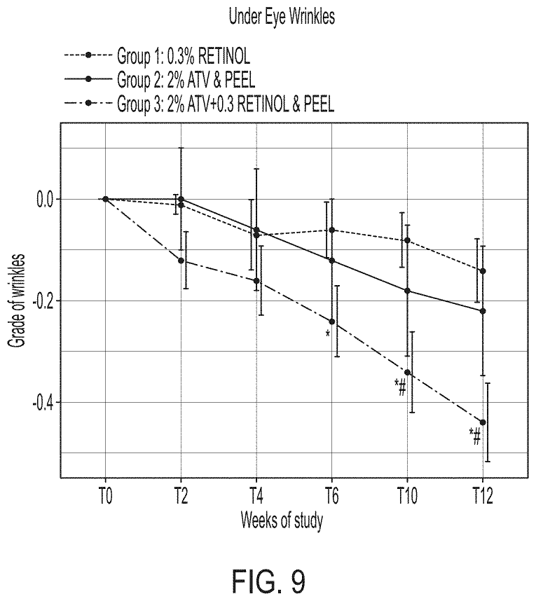

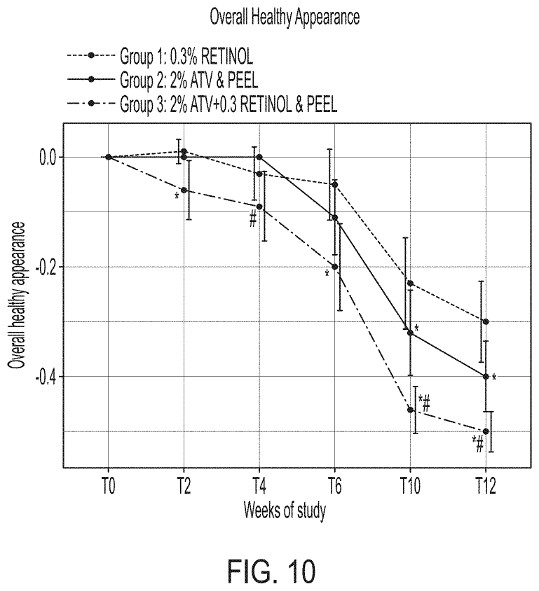

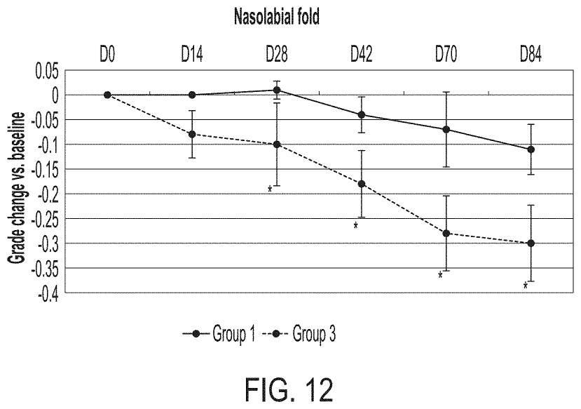

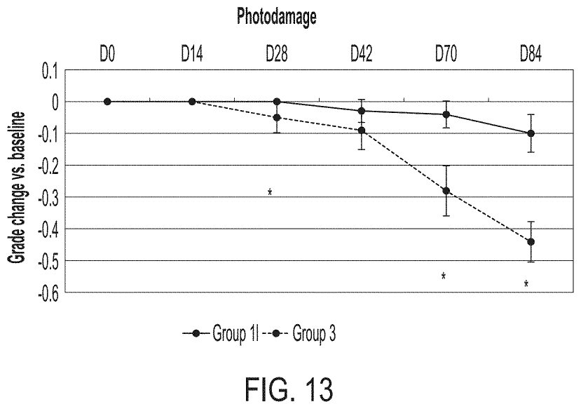

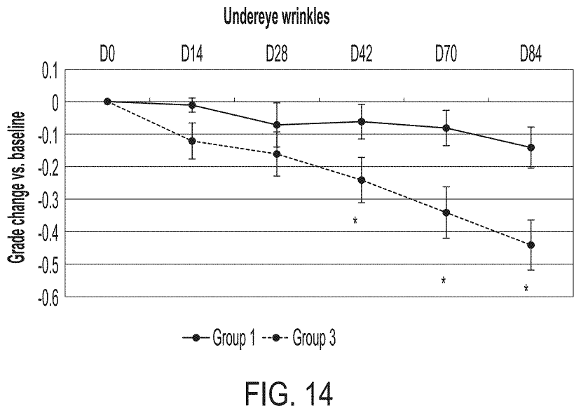

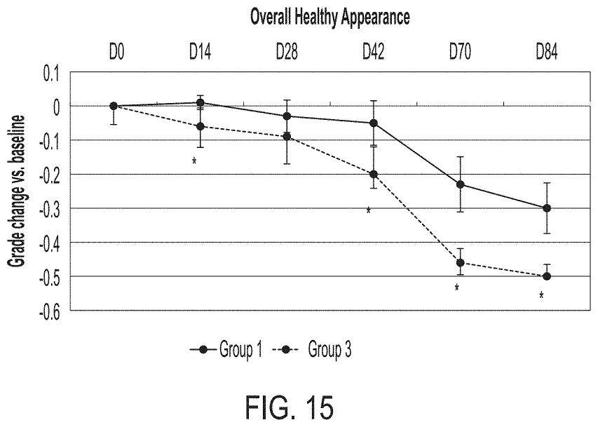

Clinical evaluation of anti-aging parameters demonstrated significant improvement over baseline for all three skincare regimen groups at Week 2 and 4 for global fine lines (FIG. 6) (or FIG. 11). By Week 6, Group 2 and 3, which included the home peel, showed a significant improvement in under eye wrinkles/crow's feet and nasolabial fold wrinkles appearance from baseline (FIGS. 7 and 9) (or FIGS. 12 and 14). By Week 12, all treatment groups demonstrated a significant improvement in all parameters assessed over baseline, except for Group 1 showing no relevant change in global wrinkles appearance, as illustrated in (FIGS. 6-10) (or FIGS. 11-15).

Treatment comparisons in the Figures demonstrated that Group 3 (home peel and acetyl trifluoromethylphenyl valylglycine+retinol-containing product), was significantly better at improving global fine lines and overall healthy appearance than Group 1 (retinol-containing product) by Week 2, and Group 2 (home peel and acetyl trifluoromethylphenyl valylglycine—containing product) for fine lines. Group 3 significantly outperformed Group 1 by Week 12 in all assessed anti-aging parameters (FIGS. 6-10) (or FIGS. 11-15).

These results demonstrate that, surprisingly, anti-aging routine containing acetyl trifluoromethylphenyl valylglycine+at least one retinoid+at least one additional chemical peel delivers superior anti-aging and skin quality benefits in an accelerated kinetic manner, and additional provides superior tolerance as compared to retinol use alone.

1-112. (canceled)

113. A method for treating skin, the method comprising:
(1) a phase (1) comprising applying to the skin:
(a) acetyl trifluoromethylphenyl valylglycine at least one time per day;
(b) at least one retinoid or derivative thereof at least one time every two to three days; and
(c) optionally, at least one additional skin peeling agent other than retinoids or derivatives thereof at least one time every two to three days,
wherein the (b) at least one retinoid or derivative thereof and the (c) optional at least one additional skin peeling agent are separately applied to the skin at least about 6 hours apart; and
wherein phase (1) lasts for a period of time of at least about 10 days; and
(2) a phase (2) comprising applying to the skin:
(a) acetyl trifluoromethylphenyl valylglycine at least one time per day; and
(b) at least one retinoid or derivative thereof at least one time every two to three days;
wherein phase (2) lasts for a period of time of at least about 5 days, and
wherein phase (2) occurs before or after phase (1).

114. The method of claim 113, wherein in phase (1), phase (2), or both, the method comprises applying to the skin (a) a composition comprising acetyl trifluoromethylphenyl valylglycine in amounts independently ranging from about 0.001 to about 25 wt %, relative to the total weight of the composition in which it is present.

115. The method of claim 113, wherein in phase (1), phase (2), or both, the (b) at least one retinoid or derivative thereof is independently chosen from retinol, retinal, retiferol, all-trans-retinoic acid, isotretinoin, 9-cis-retinoic acid, etretinate, acitretin, tazarotene, bexarotene, Adapalene, N,N-diethylaminoethyl 13-cis-retinoate, 13-cis-retinoic acid, N,N-diethylaminoethyl all-trans-retinoate, retinyl N,N-dimethyl-2-aminoacetate, retinyl palmitate, retinoid metabolites, or mixtures thereof.

116. The method of claim 113, wherein in phase (1), phase (2), or both, the (b) at least one retinoid or derivative thereof comprises retinol.

117. The method of claim 113, wherein in phase (1), phase (2), or both, the method comprises applying to the skin (b) a composition comprising at least one retinoid or derivative thereof in amounts independently ranging from about 0.0001 to about 10 wt %, relative to the total weight of the composition in which it is present.

118. The method of claim 113, wherein in phase (1), the method comprises applying (c) at least one additional skin peeling agent chosen from α-hydroxy acids, B-hydroxy acids, or mixtures thereof, to the skin.

119. The method of claim 113, wherein in phase (1), the method comprises applying (c) a composition comprising (i) at least one α-hydroxy acid chosen from lactic acid, glycolic acid, citric acid, malic acid, tartric acid, mandelic acid, derivatives thereof, or mixtures thereof, and (ii) at least one B-hydroxy acid chosen from salicylic acid, derivatives thereof, or mixtures thereof, to the skin.

120. The method of claim 113, wherein in phase (1), the method comprises applying to the skin (c) a composition comprising at least one additional skin peeling agent chosen from α-hydroxy acids, wherein the total amount of α-hydroxy acids ranges from about 0.1 to about 25 wt %, relative to the total weight of the composition in which it is present.

121. The method of claim 113, wherein in phase (1), the method comprises applying to the skin (c) a composition comprising at least one β-hydroxy acid in an amount ranging from about 0.01 to about 2 wt %, relative to the total weight of the composition in which it is present.

122. The method of claim 113, wherein in phase (1), the method comprises applying to the skin (c) a composition comprising from about 0.05 to about 1.5 wt % salicylic acid and from about 5 to about 15 wt % lactic acid, relative to the total weight of the composition in which the salicylic acid and lactic acid are present.

123. The method of claim 113, wherein the method is a method for stimulating skin renewal, a method for stimulating skin peeling, a method for reducing the appearance of nasolabial folds, a method for reducing the appearance of wrinkles, a method for reducing the appearance of discoloration of the skin, or a method for improving the appearance of the skin.

124. The method of claim 113, wherein in phase (1), phase (2), or both, the (a) acetyl trifluoromethylphenyl valylglycine is applied to the skin at least two times per day, wherein the second application is at least about 6 hours after the first application.

125. The method of claim 113, wherein in phase (1), phase (2), or both, the acetyl trifluoromethylphenyl valylglycine is applied to the skin at least one time per day, for at least 70 consecutive days.

126. The method of claim 113, wherein in phase (1), phase (2), or both, the at least one retinoid or derivative thereof is applied to the skin at least every other day, for at least 70 consecutive days.

127. The method of claim 113, wherein in phase (1), the (c) skin peeling agent is applied to the skin at least every other day for a period of at least 25 days, followed by a period of at least 10 days wherein the skin peeling agent is not applied.

128. The method of claim 113, wherein the at least one retinoid or derivative thereof and the at least one additional skin peeling agent are not applied to skin on the same day.

129. The method of claim 113, wherein phase (1) lasts for a period of time of at least about 21 days, and wherein phase (2) lasts for a period of time of at least about 10 days.

130. The method of claim 113, comprising:
(1) a phase (1) comprising applying to the skin:
(a) from about 0.01 to about 20 wt % of acetyl trifluoromethylphenyl valylglycine, at least one time per day;
(b) from about 0.0001 to about 10 wt % of at least one retinoid or derivative thereof, at least one time every two to three days; and
(c) from about 0.1 to about 25 wt % of at least one additional skin peeling agent chosen from α-hydroxy acids, β-hydroxy acids, or mixtures thereof, at least one time every two to three days,
wherein the (b) at least one retinoid or derivative thereof is optionally present in a composition that additionally comprises from about 0.01 to about 20 wt % of acetyl trifluoromethylphenyl valylglycine;
wherein the (b) at least one retinoid or derivative thereof and the (c) optional at least one additional skin peeling agent are separately applied to the skin at least about 6 hours; and
wherein phase (1) lasts for a period of time of at least about 10 days; and
(2) a phase (2) comprising applying to the skin:
(a) from about 0.01 to about 20 wt % of acetyl trifluoromethylphenyl valylglycine, at least one time per day; and
(b) from about 0.0001 to about 10 wt % of at least one retinoid or derivative thereof, at least one time every two to three days;
wherein the (b) composition comprising at least one retinoid or derivative thereof optionally additionally comprises from about 0.01 to about 20 wt % of acetyl trifluoromethylphenyl valylglycine; and
wherein phase (2) lasts for a period of time of at least about 5 days;
wherein the amount of each component is relative to the total weight of the composition in which it is present.

131. A method for treating skin, the method comprising applying to the skin:
(a) acetyl trifluoromethylphenyl valylglycine;
(b) at least one retinoid or derivative thereof; and
(c) optionally at least one additional skin peeling agent,
wherein the (a) acetyl trifluoromethylphenyl valylglycine is applied to the skin in an amount effective to reduce skin intolerance toward retinoids.

132. A kit comprising:
(i) a first container or compartment comprising a composition comprising acetyl trifluoromethylphenyl valylglycine;
(ii) a second container or compartment comprising a composition comprising at least one retinoid or derivative thereof; and
(iii) optionally a third container or compartment comprising a composition comprising at least one additional skin peeling agent other than retinoids or derivatives thereof.
